(19) United States
(12) Patent Application Publication
(10) Pub. No.: US 2014/0193846 A1
(43) Pub. Date: Jul. 10, 2014

(54) NOVEL APOC-I ISOFORMS AND THEIR USE AS BIOMARKERS AND RISK FACTORS OF ATHEROSCLEROTIC DISEASE

(71) Applicants: Ronald D. Macfarlane, Temple, TX (US); Catherine J. McNeal, Temple, TX (US); Subroto Chatterjee, Columbia, MD (US)

(72) Inventors: Ronald D. Macfarlane, Temple, TX (US); Catherine J. McNeal, Temple, TX (US); Subroto Chatterjee, Columbia, MD (US)

(73) Assignees: SCOTT & WHITE HEALTHCARE, Temple, TX (US); THE JOHNS HOPKINS UNIVERSITY, Baltimore, MD (US); THE TEXAS A&M UNIVERSITY SYSTEM, College Station, TX (US)

(21) Appl. No.: 14/112,670

(22) PCT Filed: Dec. 23, 2011
(86) PCT No.: PCT/US11/67227

§ 371 Date: Jan. 24, 2014

Related U.S. Application Data
(60) Provisional application No. 61/476,941, filed on Apr. 19, 2011.

(51) Int. Cl. G01N 33/92

(52) U.S. Cl. CPC G01N 33/92
USPC 435/11; 530/359

(57) ABSTRACT

Novel isoforms of apolipoprotein C-I (apoC-I), namely apolipoprotein C-I₁ (apoC-I₁) and apolipoprotein C-I₁′ (apoC-I₁′), both of which have a molecular weight of approximately 90 daltons greater than native apolipoprotein C-I (SEQ ID NO:6) and native apolipoprotein C-I′ (SEQ ID NO:7), are shown to be both biomarkers for diagnosing atherosclerotic disease as well as risk factors for subjects having increased risk of developing an atherosclerotic disease.

### STATEMENT AS TO FEDERALLY SPONSORED RESEARCH

This invention was made with government support under Grant No. RO1HL068794 awarded by the National Institutes of Health Heart, Lung and Blood Institute of the National Institutes of Health. The government has certain rights in the invention.

### TECHNICAL FIELD

This application relates to vascular diseases and disorders and more specifically relates to diseases and disorders wherein there is a blockage of blood vessels or reduced blood flow through blood vessels such as atherosclerotic diseases.

### BACKGROUND

Atheromas are accumulations or swellings in walls of the blood vessel and are mostly made up of macrophage cells, debris that contain lipids (cholesterol and fatty acids), calcium, and a variable amount of fibrous connective tissue (Aldons J., Nature, 407 (6801):233-241 (2000)). The accumulations are typically between the endothelium lining and the smooth muscle wall central region (media) of the arterial tube. While the early stages, based on gross appearance, have traditionally been termed “fatty streaks” by pathologists, they are not composed of adipose cells, but of accumulations of white blood cells, especially macrophages, that have taken up oxidized low-density lipoprotein (LDL). After these cells accumulate large amounts of cytoplasmic membranes (with the associated high cholesterol content) they are called “foam cells.” When foam cells die, their contents are released, which attracts more macrophages and creates an extracellular lipid core near the center to inner surface of each atherosclerotic plaque. Conversely, the outer, older portions of the plaque become more calcific, less metabolically active and more physically stiff over time. Collectively, the process of atheroma development within an individual is called atherogenesis and the overall result of the disease process is termed atherosclerosis or “hardening of the arteries.”

Atherosclerosis is an insidious and dangerous disease resulting in progressive chemical and structural injury to the blood vessels in such critical organs such as the heart, brain, and kidney. The hallmark feature of atherosclerosis is the buildup of cholesterol resulting in plaque formation, vascular remodeling, acute and chronic luminal obstruction, abnormalities of blood flow, and diminished oxygen supply to target organs. Atherosclerotic plaques can also suddenly rupture, develop a blood clot on their surface, and completely choke off a portion of heart muscle. This chain of events frequently results in heart attack or sudden death without warning. Atherosclerotic disease also predisposes people to stroke, peripheral vascular disease, lower-extremity amputation, and loss of kidney function, among other devastating outcomes (Toth, Circulation, 111:e89-e91 (2005)).

High levels of cholesterol and triglycerides have been linked to atherosclerosis. Cholesterol and triglycerides must be packaged into lipoproteins in order to circulate in the plasma, from sites of synthesis or absorption to sites of use. On the basis of their buoyant density lipoproteins are divided into 5 major classes: chylomicrons, very low density lipoproteins (VLDL), intermediate density lipoproteins (IDL), low density lipoproteins (LDL) and high density lipoproteins (HDL). LDL and HDL are known as the “bad” and “good” cholesterol, respectively. Thus, elevated levels of LDL are linked to premature development of atherosclerosis and coronary heart disease, while high levels of HDL are considered to be protective. The core of the lipoprotein, containing cholesterol ester and triglycerides, is nonpolar and hydrophobic, and the outer layer of the lipoprotein particle (containing free cholesterol, phospholipid, and specific apolipoproteins) is polarized, permitting the lipoprotein particles to be transported in the circulation. Apolipoproteins (apo) such as apolipoprotein B (apoB), apolipoprotein C (apoC) and apolipoprotein E (apoE), coat lipoprotein particles and serve a number of functions including the transport of lipids in the blood and recognition of lipoprotein particles by enzymes which process or remove lipids from the lipoprotein particles. Apolipoprotein C is a family of four low molecular weight apolipoproteins, designated as C-I, C-II, C-III, and C-IV that are surface components of chylomicrons, VLDL, and HDL (Mahley R. W. et al., J. Lipid Res., 25 (12): 1277-94 (1984)).

Reliable assessment of the separate and joint associations of major blood lipids and apolipoproteins with the risk of vascular disease is important for the development of screening and therapeutic strategies (Knopp R. H., N. Engl. J. Med., 341(7):498-511 (1999); Singh I. M. et al., JAMA, 298(7):786-798 (2007)). Landmark longitudinal population studies such as the Framingham Heart Study leave little doubt that a low high density lipoprotein-cholesterol (HDL-C) level is a risk factor for cardiovascular disease CHD (Assmann G. et al., Circulation, 109:1118-14 (2004)). Conversely, an elevated HDL-C level (>60 mg/dL) is typically associated with an attenuated risk of atherosclerosis. However, the discordance between HDL-C levels and CHD is also supported at both extremes—by the absence of CHD individuals with low HDL-C levels (e.g., ApoA-IMilano) (Sirtori C. R. et al., Circulation, 103:1949-1954 (2001)) and the presence of CHD in some but not all populations of individuals with hyperalphalipoproteinemia (HALP) (Yamashita S. et al., Atherosclerosis, 152:271-285 (2000)) leading to the conclusion that there is a wide variation in the HDL particle composition and function. In addition, the failure of torcetrapib to be effective has raised questions about the value of raising HDL-C and highlighted the need to characterize more reliably the relationship between HDL-C and vascular risk, particularly at high HDL-C levels (Barter P. J. et al., N. Engl. J. Med., 357(21):2109-2122 (2007)).

Atherosclerosis takes a huge toll on our society. According to the World Health Organization, global deaths due to cardiovascular disease are greater than any other disease. More than 81 million Americans suffer from some form of cardiovascular disease, making it the leading cause of death in the country. As of 2006, cardiovascular disease was responsible for at least one in every 2.9 deaths in the United States (American Heart Association: Heart Disease and Stroke Statistics 2010). Cardiovascular disease is the leading cause of death for both men and women in the United States (Heron M. P. et al., National Vital Statistics Reports, 57(14) (2009)). Coronary artery disease, also known as coronary heart disease, is the most common type of heart disease. In 2005, 445,687 people died from coronary heart disease (Heron M. P., National Vital Statistics Reports, 56(5) (2007)). About 47% of sudden cardiac deaths occur outside a hospital (Centers for Disease Control and Prevention, 1999, MMWR, 51(6):123-126 (2002)). This suggests that many people with heart disease do not recognize and act on early warning signs. Accordingly, there is a great need in the art to provide a quick, accurate, and effective method of identifying the risk of a vascular disease or disorder such as an atherosclerotic disease. The present application meets this need.

### SUMMARY

This application is based, at least in part, on the finding of two novel isoforms of apolipoprotein C-I (apoC-I), namely apolipoprotein C-I₁ (apoC-I₁) and apolipoprotein C-I₁′ (apoC-I₁′), both of which have a molecular weight of approximately 90 daltons greater than native apolipoprotein C-I (SEQ ID NO:6) and native apolipoprotein C-I′ (SEQ ID NO:7). These novel apoC-I isoforms can serve as biomarkers and/or risk factors for identifying subjects with the risk of developing, or having undiagnosed, atherosclerotic disease. In addition, lipoprotein (e.g., HDL) particles that contain the novel isoforms of apoC-I induce apoptosis of smooth muscle cells (e.g., aortic smooth muscle cells) in a neutral sphingomyelinase (N-SMase) dependent pathway. Furthermore, lipoprotein particles (e.g., HDL) that contain the novel isoforms of apoC-I increase nitric oxide (NO) production to toxic levels and increase the expression of cell adhesion molecules (e.g., ICAM-1, V-CAM-1, etc.) to the same extent as low density lipoprotein (LDL) in endothelial cells (e.g., aortic smooth muscle cells).

In one aspect, the application features a method of diagnosing, or determining the risk of developing, an atherosclerotic disease in a subject. The method comprises assessing a biological sample from the subject for presence of an isoform of apolipoprotein C-I comprising 20 or more consecutive amino acids of SEQ ID NO:6, wherein the isoform has a molecular weight of between 6.7 kD and 6.8 kD. The presence of the isoform in the sample indicates that the subject has an atherosclerotic disease, or is at an increased risk of developing an atherosclerotic disease compared to a subject without presence of the isoform. In certain embodiments, the isoform of apolipoprotein C-I comprises 30 or more consecutive amino acids of SEQ ID NO:6. In other embodiments, the isoform of apolipoprotein C-I comprises 40 or more consecutive amino acids of SEQ ID NO:6. In other embodiments, the isoform of apolipoprotein C-I has a molecular weight of between 6.70 kD and 6.75 kD, between 6.71 kD and 6.74 kD, or between 6.71 kD and 6.73 kD. In certain embodiments, the results of the test are communicated to the subject. In some embodiments, if the test results indicate that the subject has an atherosclerotic disease, or is at an increased risk of developing an atherosclerotic disease, the subject's medical record is modified to reflect the test results. In other embodiments, if the test results indicate that the subject has an atherosclerotic disease, or is at an increased risk of developing an atherosclerotic disease, the subject is administered a drug to reduce the symptoms of the disease, treat the disease, or reduce the likelihood of developing the atherosclerotic disease.

In a second aspect, the application provides a method of diagnosing, or determining the risk of developing, an atherosclerotic disease in a subject. The method comprises assessing a biological sample from the subject for presence of an isoform of apolipoprotein C-I comprising 20 or more consecutive amino acids of SEQ ID NO:7, wherein the isoform has the two N-terminal threonine and proline residues, and wherein the isoform has a molecular weight of between 6.5 kD and 6.55 kD. The presence of the isoform in the sample indicates that the subject has, or is at an increased risk of developing, an atherosclerotic disease compared to a subject without presence of the isoform. In certain embodiments, the isoform of apolipoprotein C-I comprises 30 or more consecutive amino acids of SEQ ID NO:7, wherein the isoform has the two N-terminal threonine and proline residues. In other embodiments, the isoform of apolipoprotein C-I comprises 40 or more consecutive amino acids of SEQ ID NO:7, wherein the isoform has the two N-terminal threonine and proline residues. In other embodiments, the isoform of apolipoprotein C-I has a molecular weight of between 6.50 kD and 6.58 kD, between 6.50 kD and 6.55 kD, or between 6.51 kD and 6.53 kD.

In another aspect, the application features a method of diagnosing, or determining the risk of developing, an atherosclerotic disease in a subject. The method comprises determining the molecular weight of apolipoprotein C-I isoforms of apolipoprotein C-I associated with a lipoprotein particle (e.g., high density lipoprotein₂ (HDL₂) or high density lipoprotein₃ (HDL₃)) in a biological sample from the subject. The method identifies a subject as having, or being at risk for developing, an atherosclerotic disease if the apolipoprotein C-I isoforms associated with a lipoprotein particle (e.g., HDL₂ or HDL₃) in the sample have a molecular weight that is 50 daltons to 150 daltons greater than native apolipoprotein C-I (SEQ ID NO:6) or native apolipoprotein C-I′ (SEQ ID NO:7), respectively. In certain embodiments, the molecular weight of apolipoprotein C-I isoforms is determined by mass spectrometry. In a specific embodiment, the mass spectrometry is performed by Matrix-assisted laser desorption/ionization (MALDI). In other embodiments, the apolipoprotein C-I isoforms associated with HDL₂ or HDL₃ in the sample have a molecular weight that is 80 daltons to 100 daltons greater than native apolipoprotein C-I (SEQ ID NO:6) or native apolipoprotein C-I′ (SEQ ID NO:7), respectively. In some embodiments, the apolipoprotein C-I isoforms associated with HDL₂ or HDL₃ in the sample have a molecular weight that is 85 daltons to 95 daltons greater than native apolipoprotein C-I or native apolipoprotein C-I′. In a specific embodiment, the apolipoprotein C-I isoforms associated with HDL₂ or HDL₃ in the sample have a molecular weight that is 90 daltons greater than native apolipoprotein C-I or native apolipoprotein C-I′.

In another aspect, the application features a method of diagnosing, or determining the increased risk of developing, an atherosclerotic disease in a subject. The method comprises assessing whether a mass spectrum for HDL apolipoprotein C-I proteins recovered from the HDL₂ or HDL₃ fractions from a biological sample from the subject comprises a peak in the mass spectrum at between 6.7 kD and 6.8 kD. The presence of the peak indicates that the subject has, or is at an increased risk of developing, an atherosclerotic disease compared to a subject without the peak in the mass spectrum at between 6.7 kD and 6.8 kD. In some embodiments, the mass spectrum includes a peak between 6.7 kD and 6.75 kD, 6.71 kD and 6.74 kD, or 6.71 kD and 6.73 kD. In another embodiment, the apolipoprotein C-I protein(s) measured by mass spectrometry is/are oxidized.

In another aspect, the application provides a method of diagnosing, or determining the increased risk of developing, an atherosclerotic disease in a subject. The method involves assessing whether a mass spectrum for HDL apolipoprotein C-I or C-I′ proteins recovered from the HDL₂ or HDL₃ fractions from a biological sample obtained from the subject comprises a peak at between 6.5 kD and 6.55 kD. The presence of the peak indicates that the subject has, or is at increased risk of developing, an atherosclerotic disease or disorder compared to a subject without the peak at between 6.5 kD and 6.55 kD. In some embodiments, the mass spectrum includes a peak between 6.51 kD and 6.55 kD, 6.51 kD and 6.54 kD, 6.52 kD and 6.54 kD, or 6.52 kD and 6.53 kD. In another embodiment, the apolipoprotein C-I protein(s) measured by mass spectrometry is/are oxidized.

In a further aspect, the application features a method of diagnosing, or determining the risk of developing, an atherosclerotic disease in a subject, the method comprising incubating lipoprotein fractions (e.g., HDL₂ or HDL₃ fractions) from a biological sample from the subject with a smooth muscle cell. The ability of the lipoprotein fractions (e.g., HDL₂ or HDL₃ sub-fractions) to cause apoptosis of the smooth muscle cell is indicative of the subject having, or being at increased risk for developing, an atherosclerotic disease. In certain embodiments, the smooth muscle cell is an aortic smooth muscle cell. In a specific embodiment, the aortic smooth muscle cell is a human aortic smooth muscle cell. In some embodiments, the HDL₂ fraction of the sample corresponds to a density range of 1.063 g/mL to 1.125 g/mL in a lipoprotein density profile. In some embodiments, the HDL₃ fraction of the sample corresponds to a density range of 1.125 g/mL to 1.210 g/mL in a lipoprotein density profile.

In yet another aspect, the application features a method of diagnosing, or determining the risk of developing, an atherosclerotic disease in a subject. The method comprises exposing endothelial cells to lipoprotein fractions (e.g., HDL₂ or HDL₃ fractions) from a biological sample from the subject. The ability of the HDL₂ or HDL₃ fractions from the subject to induce expression of nitric oxide and/or cell adhesion molecules (e.g., ICAM-1, VCAM-1, etc.) from the endothelial cells at a greater level than HDL₂ or HDL₃ fractions from a subject without the novel isoform(s) of apoC-I is indicative of the subject having, or being at increased risk for developing, an atherosclerotic disease. In certain embodiments, the endothelial cell is an aortic endothelial cell. In a specific embodiment, the endothelial cell is a human aortic endothelial cell. In some embodiments, the HDL₂ containing the novel isoforms of apoC-I corresponds to a density range of 1.063 g/mL to 1.125 g/mL in a lipoprotein density profile. In some embodiments, the HDL₃ fraction of the sample containing the novel isoform(s) of apoC-I corresponds to a density range of 1.125 g/mL to 1.210 g/mL in a lipoprotein density profile.

In all of the above aspects, in certain embodiments, the results of the test are communicated to the subject. In all of the above aspects, in some embodiments, if the test results indicate that the subject has an atherosclerotic disease, or is at an increased risk of developing an atherosclerotic disease, the subject's medical record is modified to reflect the test results. In all of the above aspects, in some embodiments, if the test results indicate that the subject has an atherosclerotic disease, or is at an increased risk of developing an atherosclerotic disease, the subject is referred to a cardiovascular specialty. In all of the above aspects, in certain embodiments, if the test results indicate that the subject has an atherosclerotic disease, or is at an increased risk of developing an atherosclerotic disease, the subject is administered a drug or combination of drugs to treat the disease, reduce the symptoms of the disease, or reduce the likelihood of developing the atherosclerotic disease. In all of the above aspects, in certain embodiments, if the test results indicate that the subject has an atherosclerotic disease, or is at an increased risk of developing an atherosclerotic disease, the subject is provided instructions for changes to diet and/or guidance regarding exercise regimens.

In another aspect, the application provides a method for identifying a compound that inhibits the development of an atherosclerotic disease and/or is useful in the treatment of an atherosclerotic disease. The method involves administering a test compound to a subject exhibiting atherosclerotic disease and obtaining a high density lipoprotein (HDL₂) or high density lipoprotein₃ (HDL₃) fraction from a biological sample from the subject. The method further involves determining the molecular weight of the apolipoprotein C-I isoforms in a lipoprotein particle (e.g., the HDL₂ or HDL₃ sub-fractions) of the sample. The compound is determined to inhibit the development of an atherosclerotic disease and/or be useful in the treatment of an atherosclerotic disease if the molecular weight of the apolipoprotein C-I isoforms in a lipoprotein particle (e.g., the HDL₂ or HDL₃ sub-fractions) of the sample is about the same molecular weight as apolipoprotein C-I isoforms in a lipoprotein particle (e.g., the HDL₂ or HDL₃ sub-fractions) of a subject without an atherosclerotic disease. The subject exhibiting an atherosclerotic disease has an HDL₂ or HDL₃ fraction that includes the novel isoforms. In some embodiments, the HDL₂ fraction of the sample corresponds to a density range of 1.063 g/mL to 1.125 g/mL in a lipoprotein density profile. In some embodiments, the HDL₃ fraction of the sample corresponds to a density range of 1.125 g/mL to 1.210 g/mL in a lipoprotein density profile.

In another aspect, the application provides a method for identifying a compound that inhibits the development of an atherosclerotic disease and/or is useful in the treatment of an atherosclerotic disease. The method involves contacting a biological sample obtained from a subject exhibiting an atherosclerotic disease with a test compound and obtaining a high density lipoprotein (HDL₂) or high density lipoprotein₃ (HDL₃) fraction from the biological sample. The method also includes the step of determining the molecular weight of the apolipoprotein C-I isoforms in the HDL₂ or HDL₃ fractions of the sample. The test compound is determined to inhibit the development of coronary artery disease and/or be useful in the treatment of atherosclerotic disease if the molecular weight of the apolipoprotein C-I or the C-I′ isoforms in the HDL₂ or HDL₃ fractions of the sample is about the same molecular weight as apolipoprotein C-I isoforms in the HDL₂ or HDL₃ fractions of a sample from a subject without an atherosclerotic disease. The subject exhibiting an atherosclerotic disease has an HDL₂ or HDL₃ fraction that includes the novel isoforms. In some embodiments, the HDL₂ fraction of the sample corresponds to a density range of 1.063 g/mL to 1.125 g/mL in a lipoprotein density profile. In some embodiments, the HDL₃ fraction of the sample corresponds to a density range of 1.125 g/mL to 1.210 g/mL in a lipoprotein density profile.

In yet another aspect, the application provides a method for identifying a compound that inhibits the development of an atherosclerotic disease and/or is useful in the treatment of an atherosclerotic disease. The method includes the steps of contacting a biological sample obtained from a subject exhibiting an atherosclerotic disease with a test compound and assessing whether a mass spectrum for HDL apolipoprotein C proteins recovered from the HDL₂ or HDL₃ fractions from the sample comprises a peak at between 6.6 kD and 6.7 kD. The presence of the peak in the mass spectrum at between 6.6 kD and 6.7 kD is indicative that the compound inhibits the development of an atherosclerotic disease and/or is useful in the treatment of an atherosclerotic disease. In some embodiments, the peak on the mass spectrum is between 6.61 kD and 6.65 kD.

In another aspect, the application provides a method for identifying a compound that inhibits the development of an atherosclerotic disease and/or is useful in the treatment of an atherosclerotic disease. The method includes the steps of contacting a biological sample obtained from a subject exhibiting an atherosclerotic disease with a test compound and assessing whether a mass spectrum for HDL apolipoprotein C proteins recovered from the HDL₂ or HDL₃ fractions from the sample comprises a peak at between 6.4 kD and 6.47 kD. The presence of the peak in the mass spectrum at between 6.4 kD and 6.47 kD is indicative that the compound inhibits the development of an atherosclerotic disease and/or is useful in the treatment of an atherosclerotic disease. In some embodiments, the peak on the mass spectrum is between 6.41 kD and 6.44 kD.

In a different aspect, methods for identifying a compound that inhibits the development of an atherosclerotic disease are provided which involves contacting a smooth muscle cell with a high density lipoprotein₂ (HDL₂) or high density lipoprotein₃ (HDL₃) fraction from a biological sample from a subject administered with a test compound. A decrease in the ability of the HDL₂ or HDL₃ fractions to cause apoptosis of the smooth muscle cell compared to HDL₂ or HDL₃ fractions from a subject having an atherosclerotic disease is indicative of the compound having the ability to inhibit the development of an atherosclerotic disease. In some embodiments, the smooth muscle cell is an aortic smooth muscle cell. In a specific embodiment, the smooth muscle cell is a human aortic smooth muscle cell. In some embodiments, the HDL₂ fraction of the sample corresponds to a density range of 1.063 g/mL to 1.125 g/mL in a lipoprotein density profile. In some embodiments, the HDL₃ fraction of the sample corresponds to a density range of 1.125 g/mL to 1.210 g/mL in a lipoprotein density profile.

In a further aspect, the application features methods for identifying a compound that inhibits the development of an atherosclerotic disease which involves contacting an endothelial cell with a high density lipoprotein₂ (HDL₂) or high density lipoprotein₃ (HDL₃) fraction from a biological sample from a subject administered with a test compound. A decrease in the ability of the HDL₂ or HDL₃ fractions to increase expression of ICAM-1 or NO in the endothelial cell compared to HDL₂ or HDL₃ fractions from a subject having an atherosclerotic disease is indicative of the compound having the ability to inhibit the development of coronary artery disease. In some embodiments, the endothelial cell is an aortic endothelial cell. In a specific embodiment, the endothelial cell is a human aortic endothelial cell. In certain embodiments, the decreased ability to induce ICAM-1 or NO expression is 3 to 5 fold. In other embodiments, the decreased ability to induce ICAM-1 or NO expression is 4 to 8 fold. In some embodiments, the HDL₂ fraction of the sample corresponds to a density range of 1.063 g/mL to 1.125 g/mL in a lipoprotein density profile. In some embodiments, the HDL₃ fraction of the sample corresponds to a density range of 1.125 g/mL to 1.210 g/mL in a lipoprotein density profile.

In all of the above aspects, in certain embodiments, the atherosclerotic disease is a coronary artery disease. In specific embodiments, the coronary artery disease is atherosclerosis-associated plaque rupture, myocardial infarction, angina, or coronary ischemia.

In all of the above aspects, in certain embodiments, the subject is a mammal. In specific embodiments, the mammal is a human.

In all of the above aspects, in certain embodiments, the biological sample is a blood sample, a plasma sample, or a serum sample. In some embodiments, the blood sample, plasma sample, or serum sample was previously obtained from the patient.

In another aspect, the invention features an isolated apolipoprotein C-I protein. This protein has 20 or more consecutive amino acids of SEQ ID NO:6 and has a molecular weight of 50 daltons to 100 daltons greater than the protein encoded by SEQ ID NO:6. In one embodiment, the molecular weight of the protein is determined by mass spectrometry. In a specific embodiment, the mass spectrometry is performed by Matrix-assisted laser desorption/ionization (MALDI) or MALDI-TOF. In certain embodiments, the protein has a molecular weight of 80 daltons to 100 daltons greater than the protein encoded by SEQ ID NO:6. In other embodiments, the protein has a molecular weight of 90 daltons greater than the protein encoded by SEQ ID NO:6. In some embodiments, the protein has a molecular weight between 6.7 kD and 6.8 kD. In other embodiments, the protein has a molecular weight between 6.71 kD and 6.75 kD.

In another aspect, the invention provides an isolated apolipoprotein C-I protein. This protein has 20 or more consecutive amino acids of SEQ ID NO:7 and has a molecular weight of 50 daltons to 100 daltons greater than the protein encoded by SEQ ID NO:7. In one embodiment, the molecular weight of the protein is determined by mass spectrometry. In a specific embodiment, the mass spectrometry is performed by Matrix-assisted laser desorption/ionization (MALDI) or MALDI-TOF. In certain embodiments, the protein has a molecular weight of 80 daltons to 100 daltons greater than the protein encoded by SEQ ID NO:7. In other embodiments, the protein has a molecular weight of 90 daltons greater than the protein encoded by SEQ ID NO:7. In some embodiments, the protein has a molecular weight between 6.5 kD and 6.57 kD. In other embodiments, the protein has a molecular weight between 6.51 kD and 6.55 kD.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can also be used in the practice or testing of the present invention. All publications, patent applications, patents, and other references mentioned herein are incorporated by reference in their entirety. The materials, methods, and examples disclosed herein are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a representative lipoprotein density profile from a subject with coronary artery disease following dextran sulfate precipitation to remove all non-HDL lipoproteins. The x-axis corresponds to the density of the lipoproteins (g/mL). The density ranges used to define HDL₂ and HDL₃ by this method are shown by the vertical lines. The y-axis corresponds to the fluorescent intensity of the lipophilic fluorophore NBD (c6-ceramide).

FIG. 2A is a representation of a MALDI-TOF mass spectrum of the HDL₂ human serum fraction from an individual without CAD.

FIG. 2B is a representation of a MALDI-TOF mass spectrum of the HDL₃ human serum fraction from an individual without CAD.

FIG. 2C is a representation of the MALDI-TOF mass spectrum of the expanded HDL₂ human serum fraction from an individual without CAD.

FIG. 2D is a representation of the MALDI-TOF mass spectrum of the expanded HDL₃ human serum fraction from an individual without CAD.

FIG. 3A is a representation of the MALDI-TOF mass spectrum of the expanded HDL₂ human serum fraction from an individual with CAD.

FIG. 3B is a representation of the MALDI-TOF mass spectrum of the expanded HDL₃ human serum fraction from an individual with CAD.

FIG. 4A is a representation of the MALDI-TOF mass spectrum of apoC-I variants (apoCI₁ and apoCI₁′) in the expanded HDL₂ human serum fraction from an individual with CAD.

FIG. 4B is a representation of the MALDI-TOF mass spectrum of apoC-I variants (apoCI₁ and apoCI₁′) in the expanded HDL₃ human serum fraction from an individual with CAD.

FIG. 5A is a representation of a DGU spectrum obtained from subject #10 (having CAD) using 60 μL of archived serum in 0.3 M Cs₂Cd(EDTA). The fluorescence intensity from NBD C6-ceramide is measured on the left axis. The vertical lines represent the cut points used to excise the HDL₂ and HDL₃ fractions. The curved line is the solute density on the right axis.

FIG. 5B is a representation of a DGU spectrum obtained from subject #10 (having CAD) using 60 μL of fresh serum in 0.3 M Cs₂Cd(EDTA). The fluorescence intensity from NBD C6-ceramide is measured on the left axis. The vertical lines represent the cut points used to excise the HDL₂ and HDL₃ fractions. The curved line is the solute density on the right axis.

FIG. 5C is a representation of a DGU spectrum obtained from subject #3 (not having CAD) using 60 μL of serum in 0.3 M Cs₂Cd(EDTA). The fluorescence intensity from NBD C6-ceramide is measured on the left axis. The vertical lines represent the cut points used to excise the HDL₂ and HDL₃ fractions. The curved line is the solute density on the right axis.

FIG. 5D is a representation of a DGU spectrum obtained from subject #1 (not having CAD) using 60 μL of serum in 0.3 M Cs₂Cd(EDTA). The fluorescence intensity from NBD C6-ceramide is measured on the left axis. The vertical lines represent the cut points used to excise the HDL₂ and HDL₃ fractions. The curved line is the solute density on the right axis.

FIG. 6 is a representation of a MALDI mass spectra of the delipidated HDL₂ fraction in the mass range containing the apoC-I isoforms from one subject without CAD (#14) and one subject with CAD (#41). The calculated mass of apoC-I is 6630.6 Da and the calculated mass of the truncated apoC-I′ is 6432.5 Da. This pattern was observed for all subjects with a normal coronary angiogram. In contrast, the spectra from subjects with multi-vessel CAD were characterized by peaks shifted approximately 90 Da higher in mass.

FIG. 7A provides a photographic representation of the effect of HDL₂ and HDL₃ on aortic smooth muscle cell (ASMC) apoptosis. Human ASMCs grown on glass cover slips were incubated with 2.5 μL of each fraction. The cells were stained with 4′,6-diamidino-2-phenylindole (DAPI) reagent. The nuclei of normal viable cells are brightly fluorescent, are ellipsoidal, have sharply defined boundaries, and are unfragmented while the nuclei of apoptotic cells are dully fluorescent, are amorphous in shape, and are fragmented as shown for subject #10.

FIG. 7B is a bar graph showing the effect of HDL₂ and HDL₃ from 4 subjects (all of whom have CAD) on ASMC apoptosis. The HDL₂ fraction stimulated apoptosis in approximately half of the cells. Error bars represent the standard deviation. Cells exposed to the medium only (control) did not exhibit any apoptosis (data not shown). *P<0.05 and **P<0.005

FIG. 8 is a bar graph showing the effect of HDL₂ on ASMC apoptosis for subjects with CAD (represented by the black bars), a subject without known CAD (dark grey bar) and subjects with a normal coronary angiogram and no CAD risk equivalent (light grey bars). The presence (+) or absence (−) of the mass shifted apoC-I and apoC-I′ ions is indicated below each subject assignment.

FIG. 9A is a photographic depiction of the effect of the control on ASMC apoptosis. The nuclei of normal viable cells are brightly fluorescent, are ellipsoidal, have sharply defined boundaries, and are unfragmented while the nuclei of apoptotic ASMCs are dully fluorescent, are amorphous in shape, and are fragmented.

FIG. 9B is a photographic depiction of the effect of the control and GW4869 on ASMC apoptosis. The nuclei of normal viable cells are brightly fluorescent, are ellipsoidal, have sharply defined boundaries, and are unfragmented while the nuclei of apoptotic ASMCs are dully fluorescent, are amorphous in shape, and are fragmented.

FIG. 9C is a photographic depiction of the effect of HDL₂ isolated from subject 10 on ASMC apoptosis. The nuclei of normal viable cells are brightly fluorescent, are ellipsoidal, have sharply defined boundaries, and are unfragmented while the nuclei of apoptotic ASMCs are dully fluorescent, are amorphous in shape, and are fragmented.

FIG. 9D is a photographic depiction of the effect of HDL₂ isolated from subject 10 and GW4869 on ASMC apoptosis. The nuclei of normal viable cells are brightly fluorescent, are ellipsoidal, have sharply defined boundaries, and are unfragmented while the nuclei of apoptotic ASMCs are dully fluorescent, are amorphous in shape, and are fragmented. Addition of the N-SMase inhibitor, GW4869, inhibits apoptosis.

FIG. 9E is a bar graph representation of the effect of GW4869 on ASMC apoptosis. The number of apoptotic cells exposed to 1 μL HDL₂ decreased from 20.1% to 1.19%. There was a doubling in the number of apoptotic cells when 2.5 μL HDL₂ was applied to the cells. *P<0.05 and **P<0.005.

FIG. 10 is a bar graph depicting the results of experiments to study the uptake of apoC-I in human ASMCs following incubation with HDL₂ fractions (identified as “b” in this figure) from subjects #170, 195, and 49. The X-axis denotes the lipoprotein fractions and the Y-axis denotes apoC-I uptake in optical density units.

FIG. 11 is a photographic depiction of the effect of HDL₂ on nitric oxide (NO) release from human aortic endothelial cells (HAECs). The left panel shows the effect of incubating HAECs with HDL₂ and the right panel shows the effect of incubating HAECs with HDL₂ and pyrrolidine dithiocarbamate (PDTC). A large amount of NO synthesis as evidenced by the fluorescence is observed in the left panel. In contrast, the production of NO was blocked when cells were pretreated with PDTC then incubated with the same HDL2 as evidenced by the barely perceptible fluorescence in the right panel.

FIG. 12 is a bar graph depicting the effects of different lipoprotein fractions on ICAM-1 expression by HAECs. The control is cell medium. The x-axis depicts the lipoprotein fractions and the y-axis depicts ICAM-1 expression in optical density units. *p<0.01 indicates statistically significant differences from control. Data are shown for a subject with apoptotic HDL (#170) where a=apoB100 containing lipoprotein fraction, b=HDL₂ and c=HDL₃ fraction, and for subject #79 who had a normally functioning HDL (i.e., non-apoptotic HDL).

### DETAILED DESCRIPTION

The present application provides novel isoforms of apolipoprotein C-I (apoC-I), namely apolipoprotein C-I₁ (apoC-I₁) and apolipoprotein C-I₁′ (apoC-I₁′). These two isoforms have a molecular weight of approximately 90 daltons greater than apolipoprotein C-I and apolipoprotein C-I′. Importantly, these novel apoC-I isoforms are found in subjects with coronary artery disease (CAD), but are generally not observed in subjects without CAD. These findings reveal a distinct difference between apoC-I spectra in these two populations of individuals and thus can serve as diagnostic biomarkers for identifying subjects having undiagnosed atherosclerotic disease as well as novel risk factors for identifying subjects having an increased risk of developing atherosclerotic disease. Lipoprotein particles (e.g., HDL) that contain the novel isoforms of apoC-I are also associated with inducing apoptosis of smooth muscle cells (SMCs) in a neutral sphingomyelinase (N-SMase) dependent pathway. These particles can induce up to a 5-fold enhancement of SMC apoptosis compared to tumor necrosis factor-α (TNF-α). In addition, HDL particles that contain the novel isoforms of apoC-I increase cell adhesion molecules (e.g., ICAM-1) production in endothelial cells to the same extent as low density lipoprotein (LDL). Furthermore, HDL particles that contain the novel isoforms of apoC-I increase the production of nitric oxide (NO) in endothelial cells. The atherogenic properties of the HDL particles are attributable to a subclass of lipoproteins enriched in at least one of the novel apoC-I isoforms.

### DEFINITIONS

The term “native apoC-I” refers to the mature 57 amino acid protein set forth in SEQ ID NO:6.

The term “native apoC-I′” refers to the truncated form of the mature apoC-I protein that is 55 amino acids in length, the sequence of which is set forth as SEQ ID NO:7.

The term ‘biological sample” means any body fluid obtained from the subject, including, but not limited to, blood, plasma, and serum. The term “subject” is a mammal, including but not limited to, a human, a chimpanzee, an orangutan, a gorilla, a monkey, a mouse, a rat, a pig, a horse, a dog, and a cow

The term “atherosclerotic disease” is any disease where there is a blockage of an artery. The atherosclerotic disease can be a disease involving the arteries that supply any territory in the body including, but not limited to, organs including the heart, the brain, the kidney, the intestines, the lung, the liver, and the reproductive system. In some embodiments, the atherosclerotic disease is peripheral arterial disease (i.e., atherosclerosis involving the arteries leading to the legs and/or the aorta (thoracic or abdominal). In specific embodiments, the atherosclerotic disease is coronary artery disease (also known as coronary heart disease). In some embodiments, the coronary artery disease is atherosclerosis-associated plaque rupture resulting in myocardial infarction, angina, or coronary ischemia, and aneurysm rupture.

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

An “isolated nucleic acid” refers to a nucleic acid that is separated from other nucleic acid molecules that are present in a naturally-occurring genome, including nucleic acids that normally flank one or both sides of the nucleic acid in a naturally-occurring genome (e.g., a yeast genome). The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., any paramyxovirus, retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

The term “exogenous” as used herein with reference to nucleic acid and a particular host cell refers to any nucleic acid that does not occur in (and cannot be obtained from) that particular cell as found in nature. Thus, a non-naturally-occurring nucleic acid is considered to be exogenous to a host cell once introduced into the host cell. It is important to note that non-naturally-occurring nucleic acids can contain nucleic acid subsequences or fragments of nucleic acid sequences that are found in nature provided that the nucleic acid as a whole does not exist in nature. For example, a nucleic acid molecule containing a genomic DNA sequence within an expression vector is non-naturally-occurring nucleic acid, and thus is exogenous to a host cell once introduced into the host cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular cell. For example, an entire chromosome isolated from a cell of yeast x is an exogenous nucleic acid with respect to a cell of yeast y once that chromosome is introduced into a cell of yeast.

“Polypeptide” and “protein” are used interchangeably herein and mean any peptide-linked chain of amino acids, regardless of length or post-translational modification. Typically, a polypeptide described herein (e.g., a apoC-I, apoC-I′, apoC-I₁, apoC-I₁′) is “isolated” when it constitutes at least 60%, by weight, of the total protein in a preparation, e.g., 60% of the total protein in a sample. In some embodiments, a polypeptide described herein consists of at least 75%, at least 90%, or at least 99%, by weight, of the total protein in a preparation.

As used herein, a “promoter” refers to a DNA sequence that enables a gene to be transcribed. The promoter is recognized by RNA polymerase, which then initiates transcription. Thus, a promoter contains a DNA sequence that is either bound directly by, or is involved in the recruitment, of RNA polymerase. A promoter sequence can also include “enhancer regions,” which are one or more regions of DNA that can be bound with proteins (namely, the trans-acting factors, much like a set of transcription factors) to enhance transcription levels of genes (hence the name) in a gene-cluster. The enhancer, while typically at the 5′ end of a coding region, can also be separate from a promoter sequence and can be, e.g., within an intronic region of a gene or 3′ to the coding region of the gene.

As used herein, “operably linked” means incorporated into a genetic construct (e.g., vector) so that expression control sequences effectively control expression of a coding sequence of interest.

### Apolipoprotein C-I

The cDNA sequence of the isolated human apoC-I gene is set forth in GenBank® Accession No. NM001645 and is also provided below (the start and stop codons are underlined and are boldened):

| (SEQ ID NO: 1) | |
|---|---|
| 1 | ctccccagcc tgataaaggt cctgcgggca ggacaggacc tcccaaccaa gccctccagc |
| 61 | aaggattcag agtgcccctc cggcctcgcc atgaggctct tcctgtcgct cccggtcctg |
| 121 | gtggtggttc tgtcgatcgt cttggaaggc ccagccccag cccaggggac cccagacgtc |
| 181 | tccagtgcct tggataagct gaaggagttt ggaaacacac tggaggacaa ggctcgggaa |
| 241 | ctcatcagcc gcatcaaaca gagtgaactt tctgccaaga tgcgggagtg gttttcagag |
| 301 | acatttcaga aagtgaagga gaaactcaag attgactcat gaggacctga agggtgacat |
| 361 | cccaggaggg gcctctgaaa tttcccacac cccagcgcct gtgctgagga ctccctccat |
| 421 | gtggccccag gtgccaccaa taaaaatcct acagaaaatt caaa 3′ |

The nucleic acid sequence encoding the apo C-I precursor polypeptide is provided below:

(SEQ ID NO: 2)
atgaggctct tcctgtcgct cccggtcctg gtggtggttc
tgtcgatcgt cttggaaggc ccagccccag cccaggggac
cccagacgtc tccagtgcct tggataagct gaaggagttt
ggaaacacac tggaggacaa ggctcgggaa ctcatcagcc
gcatcaaaca gagtgaactt tctgccaaga tgcgggagtg
gttttcagag acatttcaga aagtgaagga gaaactcaag
attgactcat ga

The nucleic acid sequence encoding the mature apo C-I polypeptide is provided below:

(SEQ ID NO: 3)
accccagacg tctccagtgc cttggataag ctgaaggagt
ttggaaacac actggaggac aaggctcggg aactcatcag
ccgcatcaaa cagagtgaac tttctgccaa gatgcgggag
tggttttcag agacatttca gaaagtgaag gagaaactca
agattgactc atga

The predicted amino acid sequence of the 83 amino acid human apoC-I precursor polypeptide is set forth in GenBank® Accession No. NP-001636 and is also provided below:

(SEQ ID NO: 4)
1 MRLFLSLPVL VVVLSIVLEG PAPAQGTPDV SSALDKLKEF
GNTLEDKARE LISRIKQSELSAKMREWFSE TFQKVKEKLK IDS 83

The apoC-I precursor polypeptide disclosed in GenBank® Accession No. NP001636 contains the signal sequence at the N-terminus. The signal sequence comprises the first 26 amino acid residues of the precursor protein and has the amino acid sequence:

(SEQ ID NO: 5)
1 MRLFLSLPVL VVVLSIVLEG PAPAQG 26

The mature apoC-I polypeptide sequence obtained by the cleavage of the signal sequence is shown below:

(SEQ ID NO: 6)
1 TPDV SSALDKLKEF GNTLEDKARE LIBRINKQSEL SAKMREWFSE
TFQKVKELK IDS 57

The contents of all of the above-referenced GenBank® records are herein incorporated by reference in their entirety in this disclosure.

ApoC-I is a 6.6 kD protein associated with triglyceride-rich lipoproteins and HDL (R. S. Shulmann et al., J. Biol. Chem., 250:182-190 (1975). Composed of 57 amino acids, its basicity and high isoelectric point are attributed to the presence of several lysine residues (M. C. Jong et al., Arterioscler. Thromb. Vasc. Biol., 19:472-484 (1999); N. S. Shacter, Curr. Opin. Lipidol., 12:297-304 (2001)). ApoC-I is a component of very-low-density lipoprotein (VLDL), intermediate density lipoprotein (IDL), and high-density lipoprotein (HDL). Apo C-I is involved in the regulation of lipase enzymes (K. Conde-Knape et al., J. Lipid Res., 43:2136-2145 (2002); J. F. Berbee et al., J. Lipid Res., 46 297-306 (2005)), and has been shown to decrease the clearance of very low density lipoproteins (VLDL) and intermediate density lipoproteins (IDL), from plasma by masking or altering the conformation of the hepatic receptor apoE (R. C. Kowal et al., J. Biol. Chem., 265:10771-10779 (1990); K. H. Weisgraber et al., J. Biol. Chem., 265:22453-22459 (1990). ApoC-I is known to be a potent stimulator of lecithin-cholesterol acyl transferase (LCAT) with approximately 78% of the activity demonstrated by apoA-I (A. K. Soutar et al., Biochemistry, 98:845-855 (1975)); it is also an inhibitor of cholesteryl ester transfer protein (CETP) (T. Gautier et al., J. Biol. Chem., 275:37504-37509 (2000); L. Dumont et al., J. Biol. Chem., 280:38108-38116 (2005)).

Isoforms of apoC-containing lipoproteins have previously been identified in biological samples as a result of glycosylation, deglycosylation, and proteolytic activity at the post-translational levels (M. Hussain et al., Biochemistry, 29:209-217 (1990); V. Zannis et al., J. Biol. Chem., 259:5495-5499 (1984); A. Scanu et al., J. Lipid Res., 25:1593-1601 (1984)). Bondarenko et al. previously identified and established that apo C-I′ is an isoform that lacks Thr-Pro residues at the N terminus of apoC-I (Int. J. Mass Spectrom. Ion Process., 219:671-680 (2002)). The amino acid sequence of the truncated isoform of apoC-I, i.e., the apo C-I′ isoform is set forth below:

(SEQ ID NO: 7)
1 DV SSALDKLKEF GNTLEDKARE LIBRINKQSEL SAKMREWFSE
TFQKVKELK IDS 55

Recently, a functional structural polymorphism of apoC-I, a T45S variant, was identified by mass spectrometry in individuals of American Indian or Mexican ancestry (M. S. Wroblewski et al., J. Lipid Res., 273:4707-4715 (2006)).

### Novel Isoforms of Apolipoprotein C-I

Two novel isoforms of apoC-I, namely apoC-I₁ and apoC-I₁′, were identified in the process of carrying out measurements of blood samples of two cohorts of normolipidemic subjects, one including subjects with no CAD, and a second cohort involving subjects with CAD. For the 10 subjects with no CAD, mass spectrometry was performed to obtain the molecular weights of proteins associated with two forms of HDL that are differentiated by their density, namely HDL₂ and HDL₃. It was found that these particles contained the known major proteins associated with these particles and that their molecular weights conformed to those provided in the scientific literature. When the measurements of the blood from the CAD subjects were performed, it was noted that there were small differences in the molecular weights of some of the proteins. Particularly striking was the mass spectrum of apoC-I, which was a major component of the mass spectrum. For all of the 10 CAD subjects, the molecular weight of the apoC-I proteins was heavier by on average 90 daltons: apoC-I₁ has a molecular weight between about 6.7 kD and about 6.8 kD; whereas the truncated version, apoC-I₁′ has a molecular weight between about 6.5 kD and about 6.6 kD. Further, there were differences in the apoC-I mass spectra of HDL₂ and HDL₃, with more modifications in the HDL₂ fraction. Specifically, both the apo C-I proteins in the HDL₂ human serum fraction measured by mass spectrometry were oxidized, whereas only the truncated apoC-I protein in the HDL₃ human serum fraction measured by mass spectrometry was oxidized. While this application is not limited by any particular mechanism of action, these results may be explained as follows: if there is no CAD, the HDL particles are not modified by the arterial wall, but if the subject has CAD, the HDL interacts with the diseased wall and is chemically modified. In support of this explanation, it is noted that HDL₂ is derived from HDL₃ in the artery as it picks up normal lipids. If the lipids are derived from the CAD region, the more vigorous chemistry taking place there will influence the HDL₃ to HDL₂ transformation. Thus, the HDL particles act as an internal probe of the health of an individual's arteries and are readily assayed by testing of a biological sample, e.g., a blood sample.

In some embodiments, the application provides apoC-I protein(s) having 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or more consecutive amino acids of SEQ ID NO:6, and having a molecular weight of 50, 55, 60, 65, 70, 75, 80, 81, 82, 83, 84, 85, 86, 87, 88, 89, 90, 91, 92, 93, 94, 95, 96, 97, 98, 99, or 100 daltons greater than the protein of SEQ ID NO:6.

In other embodiments apoC-I protein(s) are provided that have 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 or more consecutive amino acids of SEQ ID NO:6 and that have a molecular weight between 6.7 kD and 6.8 kD. In certain embodiments, the apoC-I protein(s) have a molecular weight between 6.71 kD and 6.75 kD. In other embodiments, the apoC-I protein(s) have a molecular weight between 6.71 kD and 6.73 kD. In yet other embodiments, the apoC-I protein(s) have a molecular weight of 6.71 kD, about 6.71 kD, 6.72 kD, about 6.72 kD, 6.73 kD, about 6.73 kD, 6.74 kD, or about 6.74 kD. In this context, “about” means±0.02 kD.

In some embodiments, apoC-I protein(s) are provided that have 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 or more consecutive amino acids of SEQ ID NO:6, and that have a molecular weight between 6.7 kD and 6.8 kD, and wherein the apoC-I protein as detected by mass spectrometry is oxidized.

In some embodiments, the application provides apoC-I protein(s) having 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or more consecutive amino acids of SEQ ID NO:7, wherein the apoC-I proteins have the two N-terminal Threonine and Proline residues of SEQ ID NO:7, and wherein the apoC-I protein(s) have a molecular weight of 50, 55, 60, 65, 70, 75, 80, 81, 82, 83, 84, 85, 86, 87, 88, 89, 90, 91, 92, 93, 94, 95, 96, 97, 98, 99, or 100 daltons greater than the protein of SEQ ID NO:7. In certain embodiments, the apoC-I proteins have the two N-terminal Threonine and Proline residues of SEQ ID NO:7.

In some embodiments, the application provides apoC-I protein(s) that have 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or more consecutive amino acids of SEQ ID NO:7, wherein the apoC-I proteins have the two N-terminal Threonine and Proline residues of SEQ ID NO:7, and wherein the apoC-I protein(s) have a molecular weight of between 6.50 kD and 6.58 kD. In certain embodiments, the apoC-I protein(s) have a molecular weight between 6.50 kD and 6.55 kD. In other embodiments, the apoC-I protein(s) have a molecular weight between 6.51 kD and 6.53 kD. In yet other embodiments, the apoC-I protein(s) have a molecular weight of 6.51 kD, about 6.51 kD, 6.52 kD, about 6.52 kD, 6.53 kD, about 6.53 kD, 6.54 kD or about 6.54 kD. In this context “about” means±0.02 kD.

In some embodiments, the application provides apoC-I protein(s) that have 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or more consecutive amino acids of SEQ ID NO:7, wherein the apoC-I proteins have the two N-terminal Threonine and Proline residues of SEQ ID NO:7, and that have a molecular weight between 6.50 kD and 6.58 kD, and further wherein the apoC-I protein as detected by mass spectrometry is oxidized.

### Nucleic Acids

A nucleic acid encoding an apoC-I protein (e.g., apoC-I, apoC-I′, apoC-I₁, apoC-I₁′) can have at least 70% sequence identity (e.g., 80%, 85%, 90%, 95%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or 100% sequence identity) to a nucleotide sequence set forth in SEQ ID NO:1, SEQ ID NO:2, or SEQ ID NO:3. In some embodiments, nucleic acids described herein can encode apoC-I polypeptides that have at least 70% sequence identity (e.g., 75%, 80%, 85%, 90%, 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or 100% sequence identity) to an amino acid sequence set forth in SEQ ID NOs: 4, 5, 6, or 7. For example, a nucleic acid can encode an apoC-I polypeptide having at least 90% sequence identity (e.g., 95 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or 100% sequence identity) to the amino acid sequence set forth in SEQ ID NO:4, SEQ ID NO:5, SEQ ID NO:6 or SEQ ID NO:7, or a portion thereof (e.g., a biologically-active fragment). A nucleic acid can encode an apoC-I polypeptide having at least 96% sequence identity (e.g. 96.1%, 96.2%, 96.3%, 96.4%, 96.5%, 96.6%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% sequence identity) to amino acid residues 1 to 57 of SEQ ID NO:6 or amino acid residues 1 to 55 of SEQ ID NO:7. In certain embodiments, a nucleic acid can encode an apoC-I polypeptide having at least 96% sequence identity (e.g. 96.1%, 96.2%, 96.3%, 96.4%, 96.5%, 96.6%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% sequence identity) to amino acid residues amino acid residues 1 to 57 of SEQ ID NO:6, wherein the apoC-I polypeptide has a molecular weight of between 6.6 kD and 6.7 kD. In other embodiments, a nucleic acid can encode an apoC-I polypeptide having at least 96% sequence identity (e.g. 96.1%, 96.2%, 96.3%, 96.4%, 96.5%, 96.6%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% sequence identity) to amino acid residues amino acid residues 1 to 55 of SEQ ID NO:7, wherein the apoC-I polypeptide has a molecular weight of between 6.5 kDa and 6.55 kDa.

The percent identity between a particular amino acid sequence and the amino acid sequence set forth in SEQ ID NO:4, SEQ ID NO:5, SEQ ID NO:6, or SEQ ID NO:7 is determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq-i c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences. Similar procedures can be following for nucleic acid sequences except that blastn is used.

Once aligned, the number of matches is determined by counting the number of positions where an identical amino acid residue is presented in both sequences. The percent identity is determined by dividing the number of matches by the length of the full-length apoC-I polypeptide amino acid sequence followed by multiplying the resulting value by 100. For example, an amino acid sequence that has 55 matches when aligned with the sequence set forth in SEQ ID NO:6 is 96.5 percent identical to the sequence set forth in SEQ ID NO:6 (i.e., 55÷57*100=96.5).

It is noted that the percent identity value is rounded to the nearest tenth. For example, 96.11, 96.12, 96.13, and 96.14 is rounded down to 96.1, while 96.15, 96.16, 96.17, 96.18, and 96.19 is rounded up to 96.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given apoC-I polypeptide can be modified such that optimal expression in a particular species (e.g., mammal, bacteria or fungus) is obtained, using appropriate codon bias tables for that species.

Hybridization also can be used to assess homology between two nucleic acid sequences. A nucleic acid sequence described herein, or a fragment or variant thereof, can be used as a hybridization probe according to standard hybridization techniques. The hybridization of a probe of interest (e.g., a probe containing a portion of a apoC-I nucleotide sequence) to DNA or RNA from a test source is an indication of the presence of DNA or RNA (e.g., a apoC-I nucleotide sequence) corresponding to the probe in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2× sodium chloride/sodium citrate (SSC) at 30° C., followed by a wash in 1×SSC, 0.1% SDS at 50° C. Highly stringent conditions are defined as equivalent to hybridization in 6×SSC at 45° C., followed by a wash in 0.2×SSC, 0.1% SDS at 65° C.

Isolated nucleic acid molecules encoding apoC-I polypeptides can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

### Polypeptides and Methods of Making Same

ApoC-I polypeptide candidates suitable for use herein can be identified by analysis of nucleotide and polypeptide sequence alignments. For example, performing a query on a database of nucleotide or polypeptide sequences can identify homologs and/or orthologs of apoC-I polypeptides. Sequence analysis can involve BLAST, Reciprocal BLAST, or PSI-BLAST analysis of nonredundant databases using known apoC-I amino acid sequences. Those polypeptides in the database that have greater than 40% sequence identity can be identified as candidates for further evaluation for suitability as an apoC-I polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated.

This application also provides (i) biologically active variants and (ii) biologically active fragments or biologically active variants thereof, of the apoC-I polypeptides described herein. Biologically active variants of apoC-I polypeptides can contain additions, deletions, or substitutions relative to the sequences set forth in SEQ ID NOs: 4, 5, 6, or 7. The proteins with substitutions envisaged herein will generally have not more than 50 (e.g., not more than one, two, three, four, five, six, seven, eight, nine, ten, 12, 15, 20, 25, 30, 35, 40, or 50) conservative amino acid substitutions. A conservative substitution is the substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The non-polar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a non-conservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids in SEQ ID NOs.: 4, 5, 6, or 7.

Additions (addition variants) include fusion proteins containing: (a) an apoC-I protein set forth in SEQ ID NOs: 4, 5, 6, or 7, or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagglutinin (HA), glutathione-S-transferase (GST), or maltose-binding protein (MBP)). Heterologous sequences also can be proteins useful as diagnostic or detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or endoplasmic reticulum or Golgi apparatus retention signals. Heterologous sequences can be of varying length and in some cases can be a longer sequences than the full-length target proteins to which the heterologous sequences are attached.

Biologically active fragments or biologically active variants of the apoC-I proteins have at least 40% (e.g., at least: 50%; 60%; 70%; 75%; 80%; 85%; 90%; 95%; 97%; 98%; 99%; 99.5%, or 100% or even greater) of the apoC-I activity (e.g., inhibition of CETP; inhibition of SR-B1) of the wild-type, full-length, mature protein.

The apoC-I nucleic acid and protein sequences described herein can be used to produce proteins. The methods can be performed in vitro or in vivo. To recombinantly produce an apoC-I protein, a vector is used that contains a promoter operably linked to nucleic acid encoding an apoC-I protein polypeptide. Expression vectors can be introduced into host cells (e.g., by transformation or transfection) for expression of the encoded polypeptide, which then can be purified. Expression systems that can be used for small or large scale production of apoC-I polypeptides include, without limitation, microorganisms such as bacteria (e.g., E. coli) transformed with recombinant bacteriophage DNA, plasmid DNA, or cosmid DNA expression vectors containing the nucleic acid molecules, and fungi (e.g., S. cerevisiae, Yarrowia lipolytica, Arxula adeninivorans, Pichia pastoris, Hansenula polymorpha, or Aspergillus) transformed with recombinant fungal expression vectors containing the nucleic acid molecules. Useful expression systems also include insect cell systems infected with recombinant virus expression vectors (e.g., baculovirus) containing the nucleic acid molecules, and plant cell systems infected with recombinant virus expression vectors (e.g., tobacco mosaic virus) or transformed with recombinant plasmid expression vectors (e.g., Ti plasmid) containing the nucleic acid molecules. apoC-I polypeptides also can be produced using mammalian expression systems, which include cells (e.g., immortalized cell lines such as COS cells, Chinese hamster ovary cells, HeLa cells, human embryonic kidney 293 cells, and 3T3 L1 cells) harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids described herein.

Typically, recombinant apoC-I polypeptides are tagged with a heterologous amino acid sequence such FLAG, polyhistidine (e.g., hexahistidine), hemagglutinin (HA), glutathione-S-transferase (GST), or maltose-binding protein (MBP) to aid in purifying the protein. Other methods for purifying proteins include chromatographic techniques such as ion exchange, hydrophobic and reverse phase, size exclusion, affinity, hydrophobic charge-induction chromatography, and the like (see, e.g., Scopes, Protein Purification: Principles and Practice, third edition, Springer-Verlag, New York (1993); Burton and Harding, J. Chromatogr. A 814:71-81 (1998)).

### Antibodies

The present disclosure also provides antibodies that bind to the proteins of the invention (e.g., apoC-I₁, apoC-I₁′). In one embodiment, the antibodies are specific for the proteins of the invention; such antibodies bind apoC-I₁ and/or apoC-I₁′ but not apoC-I or apoC-I′. There is no particular restriction as to the form of the antibody of the invention and the present disclosure includes polyclonal antibodies, as well as monoclonal antibodies. The antiserum obtained by immunizing animals such as rabbits with a protein of the invention, as well polyclonal and monoclonal antibodies of all classes, human antibodies, and humanized antibodies produced by genetic recombination, are also included.

A protein of the invention that is used as a sensitizing antigen for obtaining antibodies is not restricted by the animal species from which it is derived, but is preferably a protein derived from mammals, for example, humans, mice, or rats, especially preferably from humans. Protein of human origin can be obtained by using the nucleotide sequence or amino acid sequences disclosed herein.

An intact protein or its partial peptide may be used as the antigen for immunization. As partial peptides of the proteins, for example, the amino (N)-terminal fragment of the protein, and the carboxy (C)-terminal fragment can be given. “Antibody” as used herein means an antibody that specifically reacts with the full-length or fragments of the protein. In a specific embodiment, the antibody specifically binds apoC-I₁ and/or apo-CI₁′ but not apoC-I or apoC-I′.

A gene encoding a protein of the invention or a fragment thereof is inserted into a known expression vector, and, by transforming the host cells with the vector described herein, the desired protein or a fragment thereof is recovered from outside or inside the host cells using standard methods. This protein can be used as the sensitizing antigen. Also, cells expressing the protein, cell lysates, or a chemically synthesized protein of the invention may be also used as a sensitizing antigen.

The mammal that is immunized by the sensitizing antigen is not restricted; however, it is preferable to select animals by considering the compatibility with the parent cells used in cell fusion. Generally, animals belonging to the rodentia, lagomorpha, and Primates are used. Examples of animals belonging to rodentia that may be used include, for example, mice, rats, hamsters, and such. Examples of animals belonging to lagomorpha that may be used include, for example, rabbits. Examples of animals of Primates that may be used include, for example, monkeys. Examples of monkeys to be used include the infraorder catarrhini (old world monkeys), for example, Macaca fascicularis, rhesus monkeys, sacred baboons, and chimpanzees.

Well-known methods may be used to immunize animals with the sensitizing antigen. For example, the sensitizing antigen is injected intraperitoneally or subcutaneously into mammals. Specifically, the sensitizing antigen is suitably diluted and suspended in physiological saline, phosphate-buffered saline (PBS), and so on, and mixed with a suitable amount of general adjuvant if desired, for example, with Freund's complete adjuvant. Then, the solution is emulsified and injected into the mammal Thereafter, the sensitizing antigen suitably mixed with Freund's incomplete adjuvant is preferably given several times every 4 to 21 days. A suitable carrier can also be used when immunizing and animal with the sensitizing antigen. After the immunization, the elevation in the level of serum antibody is detected by usual methods.

Polyclonal antibodies against the proteins of the present disclosure can be prepared as follows. After verifying that the desired serum antibody level has been reached, blood is withdrawn from the mammal sensitized with antigen. Serum is isolated from this blood using conventional methods. The serum containing the polyclonal antibody may be used as the polyclonal antibody, or according to needs, the polyclonal antibody-containing fraction may be further isolated from the serum. For instance, a fraction of antibodies that specifically recognize the protein of the invention may be prepared by using an affinity column to which the protein is coupled. Then, the fraction may be further purified by using a Protein A or Protein G column in order to prepare immunoglobulin G or M.

To obtain monoclonal antibodies, after verifying that the desired serum antibody level has been reached in the mammal sensitized with the above-described antigen, immunocytes are taken from the mammal and used for cell fusion. For this purpose, splenocytes can be mentioned as preferable immunocytes. As parent cells fused with the above immunocytes, mammalian myeloma cells are preferably used. More preferably, myeloma cells that have acquired the feature, which can be used to distinguish fusion cells by agents, are used as the parent cell.

The cell fusion between the above immunocytes and myeloma cells can be conducted according to known methods, for example, the method by Milstein et al. (Galfre et al., Methods Enzymol. 73:3-46, 1981).

The hybridoma obtained from cell fusion is selected by culturing the cells in a standard selection medium, for example, HAT culture medium (medium containing hypoxanthine, aminopterin, and thymidine). The culture in this HAT medium is continued for a period sufficient enough for cells (non-fusion cells) other than the objective hybridoma to perish, usually from a few days to a few weeks. Then, the usual limiting dilution method is carried out, and the hybridoma producing the objective antibody is screened and cloned.

Other than the above method for obtaining hybridomas, by immunizing an animal other than humans with the antigen, a hybridoma producing the objective human antibodies having the activity to bind to proteins can be obtained by the method of sensitizing human lymphocytes, for example, human lymphocytes infected with the EB virus, with proteins, protein-expressing cells, or lysates thereof in vitro and fusing the sensitized lymphocytes with myeloma cells derived from human, for example, U266, having a permanent cell division ability.

The monoclonal antibodies obtained by transplanting the obtained hybridomas into the abdominal cavity of a mouse and extracting ascites can be purified by, for example, ammonium sulfate precipitation, protein A or protein G column, DEAE ion exchange chromatography, an affinity column to which the protein of the present disclosure is coupled, and so on. An antibody of the present disclosure may be used for the purification or detection of a protein of the present disclosure. It may also be a candidate as an agonist or antagonist of a protein of the present disclosure. Furthermore, it is possible to use it in antibody treatment for diseases in which the protein is implicated. For the administration to the human body (antibody treatment), human antibodies or humanized antibodies are preferably used because of their reduced immunogenicity.

For example, a human antibody against a protein can be obtained using hybridomas made by fusing myeloma cells with antibody-producing cells obtained by immunizing a transgenic animal comprising a repertoire of human antibody genes with an antigen such as protein, protein-expressing cells, or lysates thereof (see, e.g., WO92/03918, WO93/2227, WO94/02602, WO94/25585, WO96/33735, and WO96/34096).

Other than producing antibodies using hybridoma, antibody producing immunocytes, such as sensitized lymphocytes that are immortalized by oncogenes, may also be used.

Such monoclonal antibodies can be also obtained as recombinant antibodies produced by using the genetic engineering technique (see, for example, Borrebaeck C. A. K. and Larrick, J. W., THERAPEUTIC MONOCLONAL ANTIBODIES, Published in the United Kingdom by MACMILLAN PUBLISHERS LTD (1990)). Recombinant antibodies are produced by cloning the encoding DNA from immunocytes, such as hybridoma or antibody-producing sensitized lymphocytes, incorporating into a suitable vector, and introducing this vector into a host to produce the antibody. The present disclosure encompasses such recombinant antibodies as well.

Moreover, the antibody of the present disclosure may be an antibody fragment or modified-antibody, so long as it binds to a protein of the invention. For instance, Fab, F(ab′)₂, Fv, or single chain Fv (scFv) in which the H chain Fv and the L chain Fv are suitably linked by a linker (Huston et al., Proc. Natl. Acad. Sci. USA, 85:5879-5883, 1988) can be given as antibody fragments. Specifically, antibody fragments are generated by treating antibodies with enzymes, for example, papain or pepsin. Alternatively, they may be generated by constructing a gene encoding an antibody fragment, introducing this into an expression vector, and expressing this vector in suitable host cells (see, for example, Co et al., J. Immunol., 152:2968-2976, 1994; Better et al., Methods Enzymol., 178:476-496, 1989; Pluckthun et al., Methods Enzymol., 178:497-515, 1989; Lamoyi, Methods Enzymol., 121:652-663, 1986; Rousseaux et al., Methods Enzymol., 121:663-669, 1986; Bird et al., Trends Biotechnol., 9:132-137, 1991). In one embodiment, the antibody fragments bind specifically to the proteins of the invention: such antibodies bind apoC-I₁ and/or apoC-I₁′ but not apoC-I or apoC-I′.

As modified antibodies, antibodies bound to various molecules, such as polyethylene glycol (PEG), fluorescent substances, radioactive substances, luminescent substances, enzymes, and toxins, can be used. The antibodies of the present disclosure encompass such modified antibodies as well. To obtain such a modified antibody, chemical modifications are done to the obtained antibody. These methods are already established and conventional in the field (see, e.g., U.S. Pat. Nos. 5,057,313 and 5,156,840). The “antibodies” of the present disclosure also include such conjugated antibodies.

An antibody of the present disclosure may be obtained as a chimeric antibody, comprising non-human antibody-derived variable region and human antibody-derived constant region, or as a humanized antibody comprising non-human antibody-derived complementarily determining region (CDR), human antibody-derived framework region (FR), and human antibody-derived constant region by using conventional methods. Antibodies thus obtained can be purified to uniformity. The separation and purification methods used in the present disclosure for separating and purifying the antibody may be any method usually used for proteins. For example, column chromatography, such as affinity chromatography, filter, ultrafiltration, salting-out, dialysis, SDS polyacrylamide gel electrophoresis, isoelectric focusing, and others, may be appropriately selected and combined to isolate and purify the antibodies (Antibodies: A Laboratory Manual. Ed Harlow and David Lane, Cold Spring Harbor Laboratory, 1988); however, the invention is not limited thereto. Antibody concentration of the above mentioned antibody can be assayed by measuring the absorbance, or by the enzyme-linked immunosorbent assay (ELISA), etc.

Protein A or Protein G column can be used for the affinity chromatography. Protein A column may be, for example, Hyper D, POROS, Sepharose F. F. (Pharmacia), etc. Other chromatography may also be used, for example, such as ion-exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). These may be performed on liquid-phase chromatography such as HPLC, FPLC, and so on.

Examples of methods that assay the antigen-binding activity of the antibodies of the invention include, for example, measurement of absorbance, enzyme-linked immunosorbent assay (ELISA), enzyme immunoassay (EIA), radioimmunoassay (RIA), and/or immunofluorescence. For example, when using ELISA, a protein of the invention is added to a plate coated with the antibodies of the present disclosure, and then, the objective antibody sample, for example, culture supernatants of antibody-producing cells, or purified antibodies are added. Then, secondary antibody recognizing the primary antibody, which is labeled by alkaline phosphatase and such enzymes, is added, the plate is incubated and washed, and the absorbance is measured to evaluate the antigen-binding activity after adding an enzyme substrate such as p-nitrophenyl phosphate. As the protein, a protein fragment, for example, a fragment comprising a C-terminus, or a fragment comprising an N-terminus may be used. To evaluate the activity of the antibody of the invention, BTAcore (Pharmacia) may be used.

By using these methods, the antibody of the invention and a sample presumed to contain a protein of the invention are contacted, and the protein of the invention is detected or assayed by detecting or assaying the immune complex formed between the above-mentioned antibody and the protein. A method of detecting or assaying a protein of the invention is useful in various experiments using proteins as it can specifically detect or assay the proteins.

### Diagnostic Assays

In order to determine whether there are differences in the mass spectra of the apolipoproteins for CAD and non-CAD normolipidemic cohorts, HDL₂ and HDL₃ fractions from serum were examined. The two HDL subclasses were separated and recovered by ultracentrifugation and the apolipoprotein fractions analyzed by MALDI mass spectrometry. As noted above, two new isoforms of apoC-I, about 90 Da heavier in molecular weight than the literature value, were identified in a cohort of CAD subjects with no known risk factors. However, these new isoforms were generally not observed in a matching set of non-CAD controls. Thus, the presence of one or both of these two new isoforms of apoC-I, namely apoC-I₁ and apoC-I₁′ or their oxidized versions, namely apoC-I₂ and apoC-I₂′, can serve as biomarkers of an atherosclerotic disease and/or as risk factors for atherosclerotic diseases/disorders. Accordingly, the application provides several different diagnostic assays for determining if a subject has, or is at increased (e.g., compared to a patient without atherosclerotic disease, or a historical control from a subject who never developed an atherosclerotic disease) risk of developing, an atherosclerotic disease or disorder. The diagnostic assays can be based on the molecular weight of the novel apoC-I isoforms, the ability of lipoprotein particles containing these novel isoforms to induce apoptosis of smooth muscle cells to a greater extent than lipoprotein particles containing the native apoC-I and apoC-I′ proteins, or the ability of lipoprotein particles containing these novel isoforms to induce expression of cell adhesion molecules (e.g., ICAM-1, V-CAM-1, etc.) and/or nitric oxide (NO) in endothelial cells to a greater extent than lipoprotein particles containing the native apoC-I and apoC-I′ proteins.

(i) Molecular Weight Based Assays

Methods are provided to diagnose or determine the risk of a subject developing an atherosclerotic disease. These methods are based on the detection based on the molecular weight of the novel isoforms of apoC-I that were observed in CAD cohorts. These apoC-I isoforms are about 90 daltons greater in molecular weight than the native apoC-I isoform (SEQ ID NO:6) and its truncated form, apoC-I′ (SEQ ID NO:7). The novel isoforms can have a molecular weight of 50, 55, 60, 65, 70, 75, 80, 81, 82, 83, 84, 85, 86, 87, 88, 89, 90, 91, 92, 93, 94, 95, 96, 97, 98, 99, or 100 daltons greater than the apoC-I and apoC-I′ proteins of SEQ ID NO:6 and SEQ ID NO:7, respectively. The novel apoC-I isoform, apoC-I₁, has a molecular weight between about 6.7 kD and about 6.8 kD. In certain embodiments, the apoC-I₁ protein has a molecular weight between about 6.71 kD and about 6.75 kD. In other embodiments, the apoC-I₁ protein has a molecular weight between about 6.71 kD and about 6.73 kD. In yet other embodiments, the apoC-I₁ protein has a molecular weight of 6.71 kD, about 6.71 kD, 6.72 kD, about 6.72 kD, 6.73 kD, about 6.73 kD, 6.74 kD, or about 6.74 kD. The novel apoC-I isoform, apoC-I₁′, has a molecular weight between about 6.50 kD and about 6.58 kD. In certain embodiments, the apoC-I₁′ protein has a molecular weight between about 6.50 kD and about 6.55 kD. In other embodiments, the apoC-I₁′ protein has a molecular weight between about 6.51 kD and about 6.53 kD. In yet other embodiments, the apoC-I₁′ protein has a molecular weight of 6.51 kD, about 6.51 kD, 6.52 kD, about 6.52 kD, 6.53 kD, about 6.53 kD, 6.54 kD, or about 6.54 kD. In the context of this paragraph, the term “about” means±0.02 kD.

The diagnostic methods described herein involve assessing a biological sample of a subject for the presence of either of the novel isoforms of apolipoprotein C-I or C-I′ or their oxidized variants.

One method involves determining if a biological sample of a subject contains a protein having 10 to 20, 15 to 20, 10 to 30, 20 to 30, 10 to 40, 20 to 40, 30 to 40, 10 to 50, 20 to 50, or 30 to 50, or more consecutive amino acids of SEQ ID NO:6, and also whether the protein has a molecular weight of between 6.7 kD and 6.8 kD, between 6.71 kD and 6.75 kD, or between 6.71 kD and 6.73 kD. The presence of such a protein in the sample indicates that the subject is at increased risk of developing an atherosclerotic disease. The subject has an increased risk of having or developing an atherosclerotic disease when compared to a subject a sample from whom lacks or has reduced amounts of the protein.

Another method involves determining if an antibody that binds apoC-I (SEQ ID NO: 6) binds to a protein in a biological sample of a subject, and if so, whether the protein bound by the antibody that binds apoC-I has a molecular weight of between 6.7 kD and 6.8 kD, between 6.71 kD and 6.75 kD, or between 6.71 kD and 6.73 kD. The presence of such a protein in the sample indicates that the subject has an atherosclerotic disease, or is at increased risk of developing an atherosclerotic disease. The subject has an increased risk of developing an atherosclerotic disease when compared to a subject a sample from whom lacks or has reduced amounts of the protein, or a subject who is known to not have developed an atherosclerotic disease.

Another method involves determining if a biological sample of a subject contains a protein having 10 to 20, 15 to 20, 10 to 30, 20 to 30, 10 to 40, 20 to 40, 30 to 40, 10 to 50, 20 to 50, or 30 to 50, or more consecutive amino acids of SEQ ID NO:7, and also whether the protein has a molecular weight of between 6.50 kD and 6.58 kD, between 6.50 kD and 6.55 kD, or between 6.51 kD and 6.53 kD. The presence of such a protein in the sample indicates that the subject has an atherosclerotic disease, or is at increased risk of developing an atherosclerotic disease. The subject has an increased risk of having or developing an atherosclerotic disease when compared to a subject a sample from whom lacks or has reduced amounts of the protein, or a subject who is known to not have developed an atherosclerotic disease.

A further method involves determining if an antibody that binds apoC-I′ (SEQ ID NO:7) binds to a protein in a biological sample of a subject, and if so, whether the protein bound by the antibody that binds to apoC-I′ has a molecular weight of between 6.50 kD and 6.58 kD, between 6.50 kD and 6.55 kD, or between 6.51 kD and 6.53 kD. The presence of such a protein in the sample indicates that the subject is at increased risk of developing an atherosclerotic disease. The subject has an increased risk of having or developing an atherosclerotic disease when compared to a subject a sample from whom lacks or has reduced amounts of the protein.

In some embodiments, the method involves determining the molecular weight of apolipoprotein C-I or C-I′ isoforms of apolipoprotein C-I associated with high density lipoprotein₂ (HDL₂) or high density lipoprotein₃ (HDL₃) in a biological sample from the subject. The subject is identified as being at risk for developing an atherosclerotic disease if the apolipoprotein C-I or C-I′ isoforms associated with HDL₂ or HDL₃ in the sample have a molecular weight that is 50, 55, 60, 65, 70, 75, 80, 81, 82, 83, 84, 85, 86, 87, 88, 89, 90, 91, 92, 93, 94, 95, 96, 97, 98, 99, or 100 daltons greater than native apolipoprotein C-I (SEQ ID NO:6) or native apolipoprotein C-I′ (SEQ ID NO:7), respectively.

In other embodiments, the method involves assessing whether a mass spectrum for HDL apolipoprotein C-I proteins recovered from the HDL₂ or HDL₃ fractions from a biological sample from the subject comprises a peak in the mass spectrum at between 6.7 kD and 6.8 kD, at between 6.71 kD and 6.75 kD, or at between 6.71 kD and 6.73 kD. The presence of the peak in the mass spectrum at between 6.7 kD and 6.8 kD, at between 6.71 kD and 6.75 kD, or at between 6.71 kD and 6.73 kD indicates that the subject has atherosclerotic disease or is at increased risk of developing atherosclerotic disease compared to a sample from a subject without the peak in the mass spectrum at between 6.7 kD and 6.8 kD, at between 6.71 kD and 6.75 kD, or at between 6.71 kD and 6.73 kD.

Another method is directed to assessing a mass spectrum for HDL apolipoprotein C-I or C-I′ proteins recovered from the HDL₂ or HDL₃ fractions from a biological sample obtained from the subject comprises a peak in a mass spectrum at between 6.50 kD and 6.58 kD, at between 6.50 kD and 6.55 kD, or at between 6.51 kD and 6.53 kD. The presence of the peak in the mass spectrum at between 6.50 kD and 6.58 kD, at between 6.50 kD and 6.55 kD, or at between 6.51 kD and 6.53 kD indicates that the subject has atherosclerotic disease or is at increased risk of developing atherosclerotic disease compared to a sample from a subject without the peak at between 6.50 kD and 6.58 kD, at between 6.50 kD and 6.55 kD, or at between 6.51 kD and 6.53 kD.

The molecular weights of the proteins, in the methods outlined above, can also be determined by any method including, but not limited to, mass spectrometry (MS). MS is an analytical technique that measures the mass-to-charge ratio of charged particles (Sparkman, D. O., Mass Spectrometry Desk Reference, 2nd ed. (2006), Global View Publishing, Pittsburgh, Pa., ISBN: 978-0-9660813-2-9). It can be used for determining masses of particles such as peptides and other chemical compounds. MS involves ionizing chemical compounds to generate charged molecules or molecule fragments and measurement of their mass-to-charge ratios. In a typical MS procedure a sample is loaded onto the MS instrument, and undergoes vaporization. The components of the sample are then ionized by one of a variety of methods (e.g., by impacting them with an electron beam), which results in the formation of charged particles (ions). The ions are then separated according to their mass-to-charge ratio in an analyzer by electromagnetic fields. The ions are detected, usually by a quantitative method and the ion signal is processed into mass spectra. Any method of mass spectrometry may be used including, but not limited to, electrospray ionization, matrix-assisted laser desorption/ionization (MALDI), MALDI-TOF, glow discharge, field desorption (FD), fast atom bombardment (FAB), thermospray, desorption/ionization on silicon (DIOS), Direct Analysis in Real Time (DART), atmospheric pressure chemical ionization (APCI), secondary ion mass spectrometry (SIMS), spark ionization, thermal ionization (TIMS), and Liquid chromatography-mass spectrometry (LC-MS).

In yet another method, an antibody that binds to apoC-I (SEQ ID NO:6) or apoC-I′ (SEQ ID NO:7) is used to immunopreciptate proteins bound by the antibody from a biological sample of a test subject. The immunoprecipitated proteins are resolved on a gel alongside a molecular weight standard and a native apoC-I protein from a control subject who is known to not have an atherosclerotic disease. The presence of a protein in the test subject's sample that is 50, 55, 60, 65, 70, 75, 80, 81, 82, 83, 84, 85, 86, 87, 88, 89, 90, 91, 92, 93, 94, 95, 96, 97, 98, 99, or 100 daltons greater than native apolipoprotein C-I (SEQ ID NO:6) is indicative that the test subject has or is at increased risk of developing an atherosclerotic disease.

In yet another method, an antibody that binds to native apoC-I′ (SEQ ID NO:7) is used to immunopreciptate proteins bound by the antibody from a biological sample of a test subject. The immunoprecipitated proteins are resolved on a gel alongside a molecular weight standard and a native apoC-I′ protein from a control subject who is known to not have an atherosclerotic disease. The presence of a protein in the test subject's sample that is 50, 55, 60, 65, 70, 75, 80, 81, 82, 83, 84, 85, 86, 87, 88, 89, 90, 91, 92, 93, 94, 95, 96, 97, 98, 99, or 100 daltons greater than native apolipoprotein C-I′ (SEQ ID NO:7) is indicative that the test subject has or is at increased risk of developing an atherosclerotic disease.

Immunoprecipitation may be performed using apoC-I antiserum or purified apoC-I antibodies and running the proteins on a gel with molecular weight control. Antibodies that are specific for apoC-I are well known in the art (e.g., Krul et al., J. Lipid Res., 28(7):818-27 (1987); Wong, L., J. Lipid Res., 26(7):790-6 (1985)). Non-limiting examples of apoC-I antibodies include those available from Meridian Life Sciences, 60 Industrial Park Road, Saco, Me. 04072 (Catalog Nos. K74100G; K74110G; K74110R; and K74120G). Methods of immunoprecipitating and determining the molecular weight of the immunoprecipitated protein are well known in the art (e.g., Bonifacino, J. S. et al., Immunoprecipitation: Current Protocols in Molecular Biology, 10.16.1-10.16.29 (2001); Rosenberg, Protein Analysis and Purification: Benchtop Techniques. Springer. pp. 520. ISBN 978-0-8176-4340-9 (2005)).

(ii) Ultracentrifugation Coupled with Slice Average Variance Estimation

Another method of assessing whether a subject has or is at increased risk of developing an atherosclerotic disease is by a statistical analysis (Sliced Average Variance Analysis (SAVE) and Linear Discriminate Analysis) of the ultradensity centrifugation profile (Cook R. D. et al., Aust. N Z. J. Stat., 43:147-9 (2001)). This method provides a method of determining the likelihood of the presence of the novel higher molecular weight apoC-I isoforms in a sample.

(iii) Apoptosis-Based Assays

Another method of diagnosing that a subject has an atherosclerotic disease, or determining that a subject has an increased risk of developing an atherosclerotic disease involves contacting HDL₂ or HDL₃ fractions isolated from a biological sample from the subject with a smooth muscle cell. If the HDL₂ or HDL₃ fractions cause apoptosis of the smooth muscle cell, the result is indicative of the subject having this risk factor and therefore being at risk for developing an atherosclerotic disease including an increased risk of plaque rupture which could cause myocardial infarction. In certain embodiments, the smooth muscle cell is an Aortic Smooth Muscle Cell (ASMC), a Coronary Artery Smooth Muscle Cells (CASMC), a Pulmonary Artery Smooth Muscle Cell (PASMC), an Umbilical Artery Smooth Muscle Cell (UASMC), a Tracheal Smooth Muscle Cell (TSMC), a Bronchial Smooth Muscle Cell (BSMC), a Bladder Smooth Muscle Cell (BdSMC), a Prostate Smooth Muscle Cell (PrSMC), or a Uterine Smooth Muscle Cell (HUtSMC). In specific embodiments, the smooth muscle cell is a human Aortic Smooth Muscle Cell, (hASMC), a human Coronary Artery Smooth Muscle Cell (hCASMC), a human Pulmonary Artery Smooth Muscle Cell (hPASMC), a human Umbilical Artery Smooth Muscle Cells (hUASMC), a human Tracheal Smooth Muscle Cells (hTSMC), a human Bronchial Smooth Muscle Cells (hBSMC), a human Bladder Smooth Muscle Cells (hBdSMC), a human Prostate Smooth Muscle Cell (hPrSMC), or Human Uterine Smooth Muscle Cells (HUtSMC) (see, e.g., PromoCell products). In a specific embodiment, the smooth muscle cell is an aortic smooth muscle cell.

HDL₂ and HDL₃ fractions can be isolated from a biological sample by any method known in the art. Non-limiting methods are provided in the Examples section of this application. In certain embodiments, the cut point for the HDL₂ fraction is defined by d=1.06-1.11 g/mL and the cut point for the HDL₃ fraction is defined by d=1.11-1.18 g/mL. In some embodiments, the cut point for the HDL₂ fraction is defined by d=1.063-1.125 g/mL and the cut point for the HDL₃ fraction is defined by d=1.125-1.210 g/mL. The smooth muscle cell can be from any mammal. In certain embodiments the aortic smooth muscle cell is a human cell.

Apoptosis assays are available in a variety of formats. These include, but are not limited to, caspase assays (e.g., Caspase 3 Activity Assay (Roche Applied Sciences); DCaspase-Glo™ Assay System (Promega); casPASE™ Apoptosis Assay Kit (Genotech)); TUNEL and DNA fragmentation assays (e.g., Apoptotic DNA Ladder Kit (Roche Applied Science); DeadEnd™ Fluorometric TUNEL System (Promega); Nuclear-mediated Apoptosis Kits (BioVision)); cell permeability assays (e.g., APOPercentage™ Assay (Biocolor Assays)); annexin V assays (e.g., Annexin V, Alexa Fluor® 350 conjugate (Invitrogen); Rhodamine 110, bis-(L-aspartic acid amide), trifluoroacetic acid salt (Invitrogen)); mitochondrial and ATP/ADP assays (e.g., ApoGlow Rapid Apoptosis Screening Kit (Cambrex); Mitochondrial Membrane Potential Detection Kit (Stratagene)); protein cleavage assays (e.g., M30 CytoDEATH (Roche Applied Sciences); Anti-Poly (ADP-Ribose) Polymerase (PARP) (Roche Applied Science)); and cell proliferation and senescence assays (BioVision).

(iv) Cell Adhesion Molecule Expression Assay

Another method of diagnosing or determining risk of developing an atherosclerotic disease in a test subject involves contacting HDL₂ or HDL₃ fractions from a biological sample from the test subject with an endothelial cell. The ability of the HDL₂ or HDL₃ sub-fractions to induce expression of a cell adhesion molecule (e.g., ICAM-1, V-CAM-1, etc.) at a greater level than the comparator HDL₂ or HDL₃ sub-fractions from a biological sample from a control subject without atherosclerotic disease is indicative of the subject having or being at risk for developing atherosclerotic disease. In some embodiments, the HDL₂ or HDL₃ sub-fractions induce expression of a cell adhesion molecule to about 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 fold greater level than the comparator HDL₂ or HDL₃ sub-fractions from a biological sample from a control subject without atherosclerotic disease.

The endothelial cell can be from any mammal. In some embodiments, the endothelial cell is a Dermal Microvascular Endothelial Cell (DMEC), a Dermal Blood Endothelial Cell (DBEC), a Dermal Lymphatic Endothelial Cell (hDLEC), a Bladder Microvascular Endothelial Cell (BdMEC), a Cardiac Microvascular Endothelial Cell (CMEC), a Pulmonary Microvascular Endothelial Cell (PMEC), or a Uterine Microvascular Endothelial Cells (UtMEC). In other embodiments, the endothelial cell is a human Dermal Microvascular Endothelial Cells (hDMEC), a human Dermal Blood Endothelial Cells (hDBEC), a human Dermal Lymphatic Endothelial Cells (hDLEC), a human Bladder Microvascular Endothelial Cells (HBdMEC), a human Cardiac Microvascular Endothelial Cells (HCMEC), a human Pulmonary Microvascular Endothelial Cells (HPMEC), or a human Uterine Microvascular Endothelial Cells (HUtMEC). In certain embodiments, the endothelial cell is an aortic endothelial cell. In some embodiments, the endothelial cell is a human cell. In one embodiment, the aortic endothelial cell is a human cell.

(v) Nitric Oxide Expression Assay

Yet another method of determining risk of developing an atherosclerotic disease in a test subject involves contacting HDL₂ or HDL₃ fractions from a biological sample from the test subject with an endothelial cell. The ability of the HDL₂ or HDL₃ sub-fractions to induce expression of NO at a greater level than the comparator HDL₂ or HDL₃ sub-fractions from a biological sample from a control subject without atherosclerotic disease is indicative of the subject having or being at risk for developing atherosclerotic disease.

The endothelial cell can be from any mammal. In some embodiments, the endothelial cell is a Dermal Microvascular Endothelial Cell (DMEC), a Dermal Blood Endothelial Cell (DBEC), a Dermal Lymphatic Endothelial Cell (hDLEC), a Bladder Microvascular Endothelial Cell (BdMEC), a Cardiac Microvascular Endothelial Cell (CMEC), a Pulmonary Microvascular Endothelial Cell (PMEC), or a Uterine Microvascular Endothelial Cells (UtMEC). In other embodiments, the endothelial cell is a human Dermal Microvascular Endothelial Cells (hDMEC), a human Dermal Blood Endothelial Cells (hDBEC), a human Dermal Lymphatic Endothelial Cells (hDLEC), a human Bladder Microvascular Endothelial Cells (HBdMEC), a human Cardiac Microvascular Endothelial Cells (HCMEC), a human Pulmonary Microvascular Endothelial Cells (HPMEC), or a human Uterine Microvascular Endothelial Cells (HUtMEC). In certain embodiments, the endothelial cell is an aortic endothelial cell. In some embodiments, the endothelial cell is a human cell. In one embodiment, the aortic endothelial cell is a human cell.

### Screening Assays

Methods are provided to identify agents that are useful in treating an atherosclerotic disease. These agents include, but are not limited to, chemical compounds, proteins or peptides (e.g., antibodies or antigen-binding fragments thereof), and nucleic acid molecules (e.g., microRNAs, antisense RNAs, and ribozymes). The screening methods include assessing the effect of a test agent on the presence and/or levels of the novel apoC-I isoforms; apoptosis of smooth muscle cells; or expression of cellular adhesion molecules (e.g., ICAM-1, V-CAM-1, etc.) and/or expression of NO in endothelial cells. Such methods are described in greater detail below.

(i) Molecular Weight Based Assays

Methods are provided to identify agents that can treat an atherosclerotic disease in a subject in need thereof, or inhibit the risk of a subject developing an atherosclerotic disease. These methods are based on detecting the presence and/or levels of the novel isoforms of apoC-I that were observed in CAD cohorts.

These apoC-I isoforms are about 90 daltons greater in molecular weight than the native apoC-I isoform (SEQ ID NO:6) and its truncated form, apoC-I′ (SEQ ID NO:7). Accordingly, in one aspect the method involves administering a test compound to a subject exhibiting an atherosclerotic disease and determining from a biological sample obtained post-treatment if the apoC-I₁ and/or apoCI₁′ isoforms are present, and if present, whether they are levels lower than prior to treatment with the test compound. The subject can be any mammal (e.g., human, dog, cat, rat, mouse, pig, chimpanzee). A biological sample may be obtained prior to the treatment. In addition, one or more biological samples (e.g., blood, serum, plasma) can be obtained at several time points after the test compound has been administered. In some instances, the compound is administered for a period of time (e.g., once every day, or once every 2-3 days, or once every week, or once every month). In such instances, the biological sample can be obtained prior to each administration and at any point after administration of the test compound. If the novel apoC-I isoforms are either absent or reduced in their levels in the biological sample obtained post-administration of the compound compared to the level of these proteins prior to administration of the test compound, the test compound is adjudged to be useful in treating or inhibiting the development of an atherosclerotic disease. The presence of the novel apoC-I isoforms can easily be determined using any method including, but not limited to, mass spectrometry or immunological methods (e.g., immunoprecipitation by an apoC-I antibody and comparing the mobility of the immunoprecipitated protein with apoC-I obtained from a control, subject who has no atherosclerotic disease or known risk of developing same).

In some embodiments of this aspect, the methods involve obtaining a high density lipoprotein₂ (HDL₂) or high density lipoprotein₃ (HDL₃) fraction from a biological sample from the subject after administration of the test compound. The biological sample may be obtained within minutes, hours, days, or months after administration of the test compound. Thus, a biological sample may be obtained from a subject previously administered with the test compound. The molecular weight of the apolipoprotein C-I isoforms in the HDL₂ or HDL₃ fractions of the sample is then determined. The test compound is determined to inhibit the development of an atherosclerotic disease and/or be useful in the treatment of an atherosclerotic disease if the molecular weight of the apolipoprotein C-I isoforms in the HDL₂ or HDL₃ fractions of the sample is about the same molecular weight as apolipoprotein C-I isoforms in the HDL₂ or HDL₃ sub-fractions of a subject without atherosclerotic disease. By “about the same molecular weight” is meant a difference of ±5 kD in the molecular weight of the identified protein from the native apoC-I proteins.

In another aspect, the method is performed by contacting a biological sample obtained from a subject exhibiting coronary artery disease with a test compound and determining if the apoC-I₁ and/or apoCI₁′ isoforms of apoC-I are present, and if present, whether they are levels lower than prior to treatment of the sample with the test compound. If the novel apoC-I isoforms are either absent or reduced in their levels in the biological sample obtained post-treatment compared to the level of these proteins prior to treatment, the test compound is adjudged to be useful in treating or inhibiting the development of an atherosclerotic disease.

In some embodiments of this aspect, the methods involve obtaining a high density lipoprotein₂ (HDL₂) or high density lipoprotein₃ (HDL₃) fraction from the biological sample after administration of the test compound. The molecular weight of the apolipoprotein C-I isoforms in the HDL₂ or HDL₃ fractions of the sample is then determined. The test compound is determined to inhibit the development of an atherosclerotic disease and/or be useful in the treatment of an atherosclerotic disease if the molecular weight of the apolipoprotein C-I isoforms in the HDL₂ or HDL₃ fractions of the sample is about the same molecular weight as apolipoprotein C-I isoforms in the HDL₂ or HDL₃ fractions of a biological sample from an angiographically-normal subject. By “about the same molecular weight” is meant a difference of ±5 kD in the molecular weight of the identified protein from the native apoC-I proteins.

Another method for identifying a compound that inhibits the development of an atherosclerotic disease and/or is useful in the treatment of an atherosclerotic disease involves contacting a biological sample obtained from a subject exhibiting coronary artery disease with a test compound and assessing whether a mass spectrum for HDL apolipoprotein C proteins recovered from the HDL₂ or HDL₃ fractions from the sample comprises a peak at between 6.6 kD and 6.7 kD. The presence of the peak at between 6.6 kD and 6.7 kD identifies the compound as being useful to inhibit the development of an atherosclerotic disease and/or being useful in the treatment of an atherosclerotic disease.

A different method for identifying a compound that inhibits the development of an atherosclerotic disease and/or is useful in the treatment of an atherosclerotic disease involves contacting a biological sample obtained from a subject exhibiting coronary artery disease with a test compound and assessing whether a mass spectrum for HDL apolipoprotein C proteins recovered from the HDL₂ or HDL₃ fractions from the sample comprises a peak at between 6.5 kD and 6.55 kD. The presence of the peak at between 6.5 kD and 6.55 kD identifies the compound as being useful to inhibit the development of an atherosclerotic disease and/or being useful in the treatment of an atherosclerotic disease.

(ii) Apoptosis-Based Assays

As noted above, HDL₂ or HDL₃ fractions obtained from CAD subjects induce apoptosis of aortic smooth muscle cells to a far greater extent (about 5 to 8 fold greater level) than HDL₂ or HDL₃ fractions obtained from CAD subjects. This finding paves the way for another method for identifying a compound that inhibits the development of an atherosclerotic disease or that can be used to treat a subject having an atherosclerotic disease. In this method, a subject is administered a test compound and post-administration a biological sample is obtained from the subject. The biological sample may be obtained immediately after, minutes after, hours after, days after, months after, or even years after the administration of the test compound. HDL₂ and/or HDL₃ fractions from the biological sample are then used to contact smooth muscle cells. If the HDL₂ or HDL₃ fractions obtained from the subject after administration of the test compound decrease apoptosis of the smooth muscle cells relative to apoptosis mediated by HDL₂ or HDL₃ fractions from the same subject prior to administration of the compound, or compared to a sample obtained from the subject prior to administration of the test compound, or compared to a different subject having coronary artery disease, then the test compound is considered to be useful in inhibiting the development of an atherosclerotic disease or to be useful in treating an atherosclerotic disease.

This method can, of course, be run using a biological sample obtained from a subject who has been previously administered the test compound.

(iii) Cellular Adhesion Molecule-Expression Assay

HDL₂ or HDL₃ fractions obtained from atherosclerotic disease subjects induce expression of cellular adhesion molecules in endothelial cells to a far greater extent than HDL₂ or HDL₃ fractions obtained from a control subject without atherosclerotic disease. In some embodiments, the HDL₂ or HDL₃ sub-fractions from atherosclerotic subjects induce expression of a cell adhesion molecule to about 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 fold greater level than the comparator HDL₂ or HDL₃ sub-fractions from a biological sample from a control subject without atherosclerotic disease. This finding provides another method for identifying a compound that inhibits the development of an atherosclerotic disease or that can be used to treat a subject having an atherosclerotic disease. In this method, a biological sample is obtained from a subject with an atherosclerotic disease (e.g., CAD) treated with a test compound. The biological sample may be obtained immediately after, minutes after, hours after, days after, months after, or even years after the administration of the test compound. HDL₂ and/or HDL₃ fractions from the biological sample are then used to contact endothelial cells. If the HDL₂ or HDL₃ fractions obtained from the subject treated with a test compound decrease expression of a cell adhesion molecule (e.g., ICAM-1, V-CAM-1, etc.) on the endothelial cells relative to expression of ICAM-1 on endothelial cells treated with HDL₂ or HDL₃ fractions from the same subject prior to administration of the compound, or compared to expression of a cell adhesion molecule on endothelial cells treated with HDL₂ or HDL₃ fractions obtained from different subject having coronary artery disease, then the test compound is considered to be useful in inhibiting the development of an atherosclerotic disease or to be useful in treating an atherosclerotic disease.

(iv) Nitric Oxide (NO) Expression Assay

HDL₂ or HDL₃ fractions obtained from CAD subjects induce expression of NO in endothelial cells to a far greater extent than HDL₂ or HDL₃ fractions obtained from subjects without CAD. In some embodiments, the HDL₂ or HDL₃ sub-fractions induce expression of NO to about 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 fold greater level than the comparator HDL₂ or HDL₃ sub-fractions from a biological sample from a control subject without atherosclerotic disease. This finding provides yet another method for identifying a compound that inhibits the development of an atherosclerotic disease or that can be used to treat a subject having an atherosclerotic disease. In this method, a biological sample is obtained from a subject with an atherosclerotic disease (e.g., CAD) treated with a test compound. The biological sample may be obtained immediately after, minutes after, hours after, days after, months after, or even years after the administration of the test compound. HDL₂ and/or HDL₃ fractions from the biological sample are then used to contact endothelial cells. If the HDL₂ or HDL₃ fractions obtained from the subject treated with a test compound decrease expression of NO in the endothelial cells relative to expression of NO in endothelial cells treated with HDL₂ or HDL₃ fractions from the same subject prior to administration of the compound, or compared to expression of NO in endothelial cells treated with HDL₂ or HDL₃ fractions obtained from different subject having coronary artery disease, then the test compound is considered to be useful in inhibiting the development of an atherosclerotic disease or to be useful in treating an atherosclerotic disease.

The following examples are intended to illustrate, not limit, the invention.

### EXAMPLES

### Example 1

### Materials and Methods

The following materials and methods were used in Examples 2-4.

### Materials and Reagents

The dicesium-cadmium-EDTA complex (Cs₂CdEDTA) was synthesized in the laboratory following a published protocol (J. D. Johnson et al., Anal. Chem., 77:7054-7061 (2005)). The fluorophore 6-((N-(7-nitrobenz-2-oxa-1,3-diazol-4-yl)amino)-hexanoyl) sphingosine, NBD (c₆-ceramide), was obtained from Molecular Probes (Eugene, Oreg.). EDTA, sinapinic acid, Dextralip® 50, magnesium chloride hexahydrate, and trifluoroacetic acid (TFA) were from Sigma Aldrich (St. Louis, Mo.). Reagent-grade methanol, acetonitrile, and dimethyl sulfoxide (DMSO) were obtained from EM Science (Gibbstown, N.J., USA). Strata C18-E solid phase extraction cartridges and syringe adapter caps were purchased from Phenomenex (Torrance, Calif.). De-ionized water used in all experiments was from a Milli-Q water purification system (Millipore, Bedford, Mass.).

### Serum Collection

Serum samples were collected from all the human subjects in the fasting state into 9.5 ml Vacutainer tubes treated with polymer gel and silica activator (366510, Beckton-Dickinson Systems, Franklin Lakes, N.J.). Serum was separated from erythrocytes by centrifugation at 3200 rpm for 20 min at 5° C. The supernatant (serum) was aspirated and stored at −80° C. prior to analyses. Seven normolipidemic subjects with diagnosed coronary artery disease (CAD) and 8 non-CAD controls determined by angiography or medical record were used in this study. Serum samples were selected from the laboratory serum library, which consists of donated serum from Scott & White Hospital (Temple, Tex.) patients as part of a study of atherogenic HDL. Informed consent was obtained from all donors.

### Separation of Lipoprotein Particles by Preparative Density Gradient Ultracentrifugation

Prior to ultracentrifugation, serum was first treated with dextran sulfate and magnesium chloride to precipitate out apoB-containing lipoproteins. A 10 g/L solution containing dextran sulfate and 0.5M magnesium chloride was added to a sample of serum at a volume of 10% of the serum volume. This mixture was vortexed and allowed to sit at room temperature for 10 min. The mixture was then centrifuged by tabletop centrifuge at 12,000 rpm for 5 min. The supernatant was then prepared for ultracentrifugation. Into a 1.5 ml Eppendorf tube, 200 μl of the serum supernatant was subsequently stained (for imaging) with 10 μl of a solution of NBD in DMSO (1 mg/ml), mixed with 1100 μl 0.300M Cs₂CdEDTA, and vortexed at 1400 rpm for 1 min. This homogenous mixture was then incubated at room temperature for 30 min. A 1000 μl aliquot was transferred to a 1.5 ml thick-walled polycarbonate open-top ultracentrifuge tube (Beckman Instruments, Fullerton, Calif.). This solution was centrifuged for 6 h at 120,000 rpm and 5° C. in a Beckman Optima™ TLX-120 Ultracentrifuge with a 30° fixed angle TLA 120.2 rotor.

### Fluorescence Imaging and Generation of Lipoprotein Density Profile

A custom-built fluorescence imaging system was used to measure the distribution of the lipoprotein particles in the ultracentrifuge tube following ultracentrifugation. The light source used was a Fiber-Lite MH-100 Illuminator, (MH100A, Edmund Industrial Optics). A digital color microscope camera (S99808, Optronics, Goleta, Calif.) was used. The camera and light source were placed orthogonally to each other on an optical bench and a slit (1 cm×4 cm) was placed 8 cm away from the tube holder and suspended by a post-holder to collimate the excitation beam. A gain of 1.0000 and an exposure time of 41.1 mS were chosen using the accompanying MicroFire camera software. A blue-violet excitation filter (BG-12, Schott, Edmund Industrial Optics) with a bandwidth centered at 407 nm and a yellow emission filter (OG-515, OEM, Edmund Industrial Optics) with a bandwidth centered at 570 nm was chosen to match the NBD (c₆-ceramide) excitation and emission. From the image capture of the UC tube, a density profile was generated as described by a published method (J. D. Johnson et al., Anal. Chem., 77:7054-7061 (2005)). Using Origin 7.0 software, the image was first converted into grayscale intensity values as a function of pixels in two dimensions. Later the grayscale intensity from a small strip of 10 pixels oriented in the center of the UC tube was averaged. This average grayscale intensity was then plotted as a function of tube coordinate (0-34 mm) to give the final lipoprotein density profiles. This method also provides for the separation of lipoprotein subclasses and their subsequent recovery from serum. Excision of high density lipoprotein (HDL) fractions was achieved through the determination of the position of these fractions in an ultracentrifugation tube based upon their respective tube coordinates. The tube coordinate scale ranged from 0-34 mm which corresponds to the distance from the top to the bottom of the ultracentrifugation tube respectively. The cut points corresponded to the density ranges of 1.063-1.125 g/ml for high density lipoprotein₂ (HDL₂), and 1.125 to 1.210 g/ml for high density lipoprotein₃ (HDL₃). These defined density ranges were used to determine the cut positions for all serum samples in this study.

Recovery of HDL fractions

Following ultracentrifugation, the tubes were frozen in liquid nitrogen by lowering into a custom 10-slot holder, causing the liquid in the tubes to freeze from the bottom to the top. To cut the respective fractions a micrometer/tube holder assembly was used to dial in the correct cut points. This micrometer/tube holder assembly contains a micrometer head, which functions to advance the position of the UC tube relative to the location of the notch for the saw blade. A Dremel® scroll saw (Racine, Wis.) was fitted with 0.25 mm blades for the cutting of the tubes.

### Delipidation and Desalting of Lipoprotein Fractions

HDL fractions were desalted and delipidated using a Phenomenex C18 cartridge (Torrance, Calif.). Briefly, the HDL fractions were mixed with an equal volume of 0.1% TFA in water. This sample mixture was loaded on a cartridge that had been conditioned with 3 ml 0.1% (v/v) TFA in acetonitrile, followed by 3 ml of 0.1%(v/v) TFA in water, the cartridge was then rinsed with 3 ml 0.1%(v/v) TFA in water to remove any salts and water soluble contaminants from the ultracentrifugation spin. Proteins were eluted in 100 μl aliquots of 0.1%(v/v) TFA in acetonitrile and the first elution which contained all serum proteins, was evaporated to dryness, and reconstituted in 100 μl 0.1%(v/v) TFA in water for Matrix-assisted laser desorption/ionization (MALDI) analysis.

### MALDI-TOF Mass Spectrometry

MALDI spectra of HDL fraction proteins were recorded using an Applied Biosystems Voyager-DE STR (Foster City, Calif., USA) in linear mode at an acceleration voltage of 25 kV using a 93% transmission acceleration grid. Delay time was set at 575 ns and 100 shots per spectrum were collected. Mass ranges were acquired between 5,000 and 35,000 m/z. The system was calibrated externally with bovine insulin (MW 5733.6), bovine serum albumin (MW 66,341), and Escherichia coli thioredoxin (MW 34,622). In a 1:1 ratio, 1 μl of each sample was added to 1 μl of 10 mg/ml sinapinic acid and spotted onto a 100 spot stainless steel plate.

### Example 2

### Lipoprotein Particle Density Profile

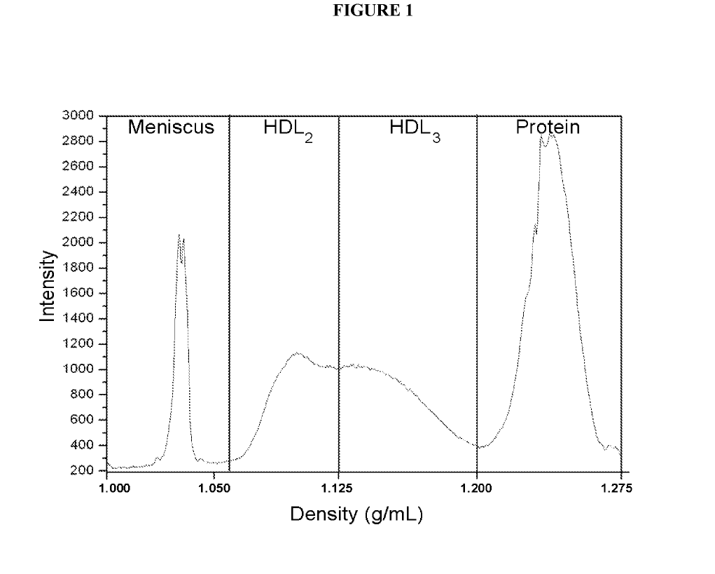

The uptake of a fluorescent probe by the lipoprotein particles allows for the visualization and identification of the distribution of lipoproteins in a lipoprotein density profile. (FIG. 1). All apoB containing lipoproteins (LDL, VLDL and TRL) were removed by dextran sulfate precipitation prior to density gradient ultracentrifugation (DGU). The first “peak” in FIG. 1 is an imaging artifact that corresponds to the reflection of fluorescence radiation emanating from the lipoprotein layers to the meniscus in the UC tube. The HDL₂ and HDL₃ particle distributions span the density range from 1.063-1.210 g/ml. The distribution above this range consists of proteins, mainly albumin, that have incorporated the fluorescent label and sediment to the bottom of the tube. This method is used to not only characterize the density of the lipoprotein particles but also to provide a means of recovering specific subclasses for further characterization. For the profile shown in FIG. 1, the cut points used to recover the HDL₂ and HDL₃ fractions are indicated by the vertical lines which correspond to 1.063-1.125 g/mL and 1.125-1.210 g/ml respectively, for HDL₂ and HDL₃. The density profiles for the two cohorts showed considerable variability in the relative intensities of the two HDL subclasses.

### Example 3

### MALDI Analysis of Non-CAD Cohort

MALDI spectra of HDL₂ and HDL₃ fraction proteins from a subject without CAD were obtained.

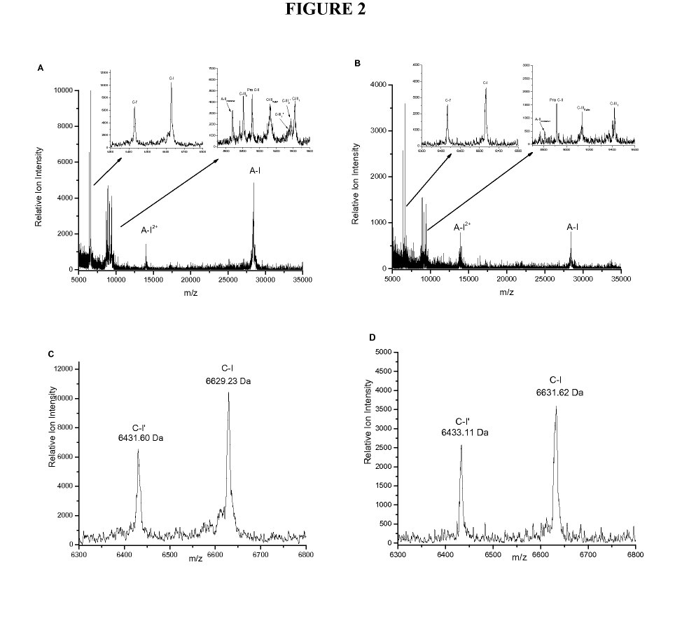

FIG. 2 shows the MALDI spectra for the HDL apolipoproteins recovered from HDL₂ and HDL₃ fractions from a subject without CAD. FIG. 2A shows the spectrum in the mass range from m/z 5,000 to 35,000 for the HDL₂ region. As shown in the lower portion of FIG. 2A, the dominant peaks in the spectrum are from apoA-I and apoC-I with significant contributions from a cluster of apo-CII, III ions in the region around m/z 9000. ApoC-1 is the dominant peak in the mass spectrum despite its factor of ten lower serum concentration relative to the dominant apoA-1. This enhancement is due to the high proton affinity of lysine-rich apoC-I in the gas phase desorption plume of the MALDI laser. Utilizing this feature, coupled with the low mass spectral background in the apoC-I region, it was elected to focus on this molecule for the initial studies.

FIG. 2B shows the corresponding mass spectra for the HDL₃ subclass fraction. The mass spectra are similar to that for HDL₂ with some differences in the apoC-II, III region but apoC-I is the dominant peak, as observed for HDL₂.

FIGS. 2C and D are an expanded versions of the apoC-I region. Two peaks were observed that were designated as apoC-I′ and apoC-I based upon their molecular weights. Bondarenko et al. previously identified and established that apo C-I′ is an isoform that lacks Thr-Pro residues at the N terminus (P. V. Bondarenko et al., Ion Process, 219:671-680 (2002)).

For the seven angiographically confirmed non-CAD cohort of patients, all spectra displayed apoC-I peaks whose masses are in accordance with the literature values. Table 1 shows the average masses and standard deviation of apoC-I′ and apoC-I for the control cohort compared to the calculated masses of these proteins. (The inherent accuracy in this mass region was determined to be ±1.2 u using an insulin standard).

TABLE 1*
Summary of m/z values for apoC-I

| | Control Cohort | Control Cohort | CAD Cohort | CAD Cohort |
|---|---|---|---|---|
| | Apo C-I′ | Apo C-I | Apo C-I₁′ | Apo C-I₁ |
| Average Mass (Da) | 6432.95 ± 8.23 | 6632.52 ± 1.98 | 6520.03 ± 16.04 | 6721.58 ± 8.10 |
| Calculated Mass | 6432.5 | 6630.6 | 6432.5 | 6630.6 |
| Difference (Da) | 0.45 ± 8.23 | 1.92 ± 1.98 | 87.53 ± 16.04 | 90.98 ± 8.10 |

*Data was collected and averaged from 16 patients.

### Example 4

### MALDI Analysis of CAD Cohort

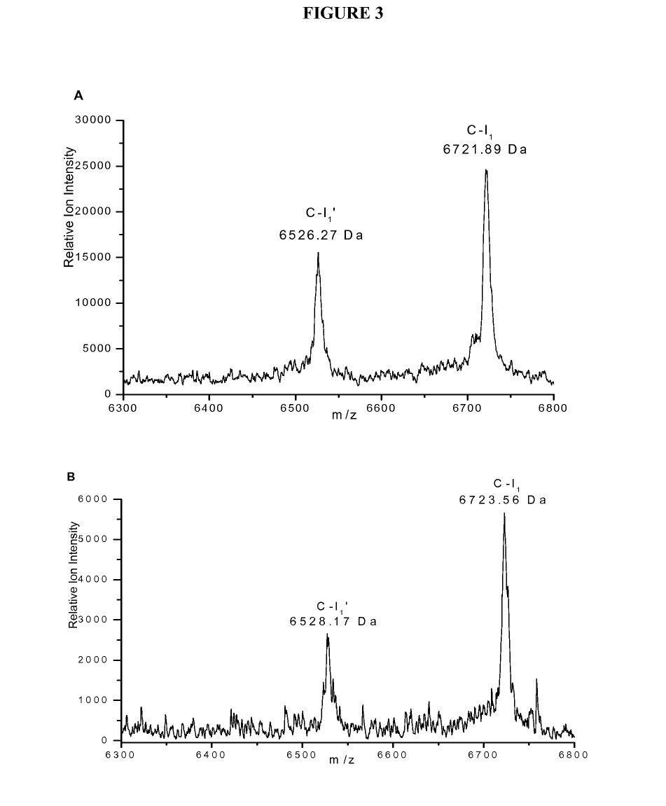

MALDI spectra of HDL₂ and HDL₃ fraction proteins from a subject with CAD were obtained. FIG. 3 provides the MALDI spectra of the apoC-I region for two subjects taken from the CAD cohort. More specifically, FIGS. 3A and 3B are the apoC-I spectra for the HDL₂ and HDL₃ subclasses, respectively. Both the full and truncated forms of apoC-I were detected but their masses were higher by approximately 90 Da compared to the controls. All 8 of the CAD cohorts were found to have these new isoforms (apoC-I₁ and truncated form apo C-I₁′). In contrast, none of the controls were found to have these new isoforms.

It is clear from this data that the new isoforms of apoC-I (apoC-I₁ and truncated form apo C-I₁′) are linked to CAD, at least for this cohort of CAD subjects. The approximate 90 Da increase for both the full apoC-I₁ and its truncated form, apo C-I₁′, indicates that a mutation of the apoC-I gene is most likely involved. If the mutation takes place at a site that is involved in the Lecithin-cholesterol acyltransferase (LCAT)/Cholesteryl ester transfer protein (CETP) activity of apoC-I within the HDL system, its role in lipoprotein metabolism could be compromised contributing to an atherogenic form of HDL.

### Example 5

### Oxidation of ApoC-I₁ and ApoC-I₁′

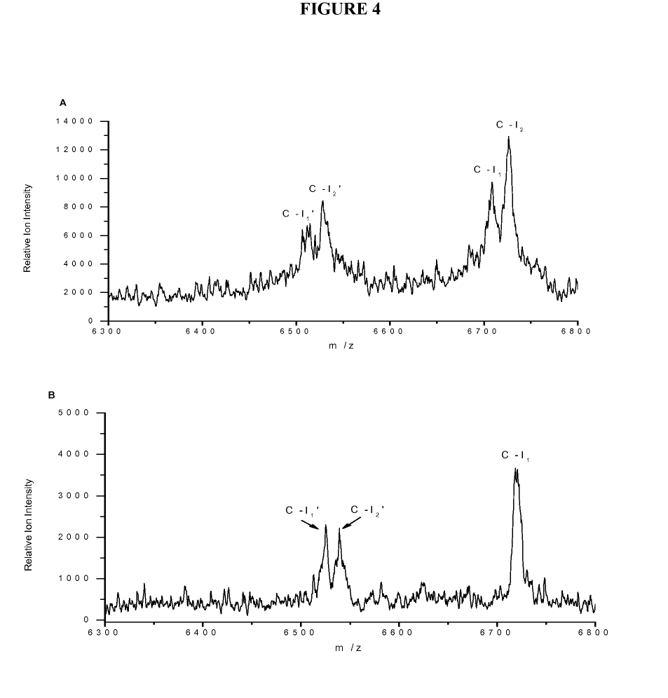

The new apoC-I isoforms (apoC-I1 and truncated form apo C-I1′) identified in CAD cohorts appear to be particularly susceptible to the oxidative stress associated with CAD. An example is shown in FIG. 4. FIG. 4A is the spectrum derived from the HDL₂ fraction showing satellite peaks (apoC-I₂ and apo-C-I₂′) 16 u higher for both the full form identified as apoC-I₁ and truncated form (apo C-I₁′). But for the HDL₃ fraction, only the truncated form has an oxidation satellite (FIG. 4B; apo-C-I2′). The apoC-I isoform spectra for the remaining CAD cohorts showed considerable variability in the degree of oxidation as well as molecular weight. This variability is reflected in the larger standard deviation of the measured average mass compared to the controls as shown in Table 1.

The pattern of oxidation of the new isoforms is particularly intriguing. Referring to FIG. 4, the apoC-I₁ isoform is not oxidized in the HDL₃ fraction but is oxidized in the truncated form (apoC-I₂′) suggesting that apoC-I₁′ more amenable to oxidation. But when the HDL₃→HDL₂ remodeling occurs, apoC-I₁ becomes more vulnerable to oxidation to apoC-I₂. For some of the CAD subjects, apoC-I₁ and apoC-I₁′ show oxidized forms in both the HDL₂ and HDL₃ subclasses.

### Example 6

### Special Case: A Person with the Risk Factor Who has not Developed CAD

One of the controls included in the study was a 56 year old subject who was initially selected as a control based primarily on having no risk factors and a sustained symptom-free healthy profile. This patient also had a family history of CAD. This case is included because it may have relevance to understanding the origin of the heavier isoforms of apoC-I. The subject's apoC-I mass spectral profile matches that of the CAD cohort shown in FIGS. 3A and 3B. In addition, the subject's HDL₂ fraction was found to induce smooth muscle apoptosis (unpublished results), a potential atherogenic property linked to apoC-I (A. Kolmakova et al., Throm. Vasc. Biol., 24:64-269 (2004)).

This “special case” supports the notion that the new isoforms of apoC-I may be linked to genetic mutation. Although the subject was asymptomatic for CAD, the signature of this new apparently atherogenic apoC-1₁ isoform was present in the HDL fractions. This feature, if driven by genetic mutation, could underlie the known family history of pre-mature CAD for this individual.

### Example 7

### Materials and Methods

The following materials and methods were used in Examples 8-16.

### Subjects

This study was approved by the Scott & White Institutional Review Board. Serum samples were obtained from 21 Caucasian subjects; 8 (5 women) had angiographically-proven multivessel CAD and a history of either percutaneous intervention and/or coronary artery bypass surgery. Another subject had a left bundle branch block but no symptomatic CAD. However this subject had moderate extracranial cerebrovascular and peripheral arterial disease. Four Caucasian women had a normal stress test, electrocardiogram (ECG) and/or echocardiogram and no history of symptomatic cardiovascular diseases (CVD) or CAD risk equivalent disease (cerebral vascular disease, peripheral vascular disease, diabetes mellitus, etc. (Grundy S. M. et al., Circulation, 110:227-239(2004)). A cohort of patients who had a normal coronary angiogram (no lesions>10%) within the six months prior to enrollment and no history of symptomatic cerebrovascular disease, peripheral arterial disease or CAD risk equivalent were recruited. 8 subjects (7 women, 1 Hispanic; 1 African-American male) with HDL-C levels comparable to the individuals with CAD irrespective of gender, age or race/ethnicity were selected. All but 6 of the 21 subjects had HDL-C levels >60 mg/dL. All but one of the 21 subjects had at least one classic risk factor including the subjects without CAD. Statin therapy was prescribed in all of the subjects with CAD and half of the subjects without CHD who also had an elevated low density lipoprotein-cholesterol (LDL-C) level. The characteristics of each subject are summarized in Table 2. Serum was obtained after a 10-12 hr. fast and stored without preservative at −80° C.

TABLE 2
Characteristics of each subject.

| | Age (yr)/ | | | | | | | | |
|---|---|---|---|---|---|---|---|---|---|
| Subject | Race and | | | | | | | | |
| ID | Gender | Angio | HDL-C | LDL-C | TC | TG | Lp(a) | C-I | A-I |
| 1 | 48/WF | Nl | 62 | 74 | 155 | 94 | | 6.3 | 157.5 |
| 3 | 77/WF | Nl | 71 | 130 | 226 | 124 | | 8.6 | 163 |
| 7 | 44/WF | Nl | 76 | 95 | 185 | 70 | | 5.3 | 170.8 |
| 10 | 79/WF | MVD | 71 | 84 | 173 | 92 | 103 | 13.3 | 153.8 |
| 13 | 66/HF | Nl | 51 | 64 | 130 | 77 | | 7.4 | 165 |
| 14 | 76/WF | Nl | 89 | 115 | 220 | 81 | | 10.9 | 152 |
| 16 | 74/WF | Nl | 42 | 145 | 223 | 179 | | 7.1 | 138 |
| 24 | 78/WF | Nl | 50 | 109 | 50 | 225 | | 10.2 | 138.8 |
| 25 | 50/BM | Nl | 64 | 75 | 64 | 160 | | 7.5 | 169 |
| 41 | 74/WM | MVD | 59 | 87 | 166 | 102 | 125 | 11.6 | 148.6 |
| 47 | 72/WF | CHD | 94 | 135 | 255 | 129 | 7 | 26.4 | 168.8 |
| | | equiv. | | | | | | | |
| 49 | 56/WF | ** | 76 | 140 | 231 | 75 | 9 | 16.5 | 155.1 |
| 79 | 40/WF | ** | 88 | 160 | 144 | 277 | 115 | 11.1 | 176.8 |
| 84 | 37/WF | MVD | 43 | 36 | 110 | 154 | 39 | 7.9 | 134.3 |
| 129 | 82/WF | *** | 61 | 72 | 166 | 165 | 32 | 10.6 | 151.1 |
| 141 | 59/WF | MVD | 41 | 76 | 156 | 193 | 77.6 | | |
| 143 | 54/WM | MVD | 70 | 72 | 151 | 45 | 227 | 10.6 | 148.5 |
| 146 | 77/WF | MVD | 86 | 85 | 189 | 92 | 14.4 | 13.6 | 162.5 |
| 170 | 75/WM | MVD | 91 | 61 | 163 | 56 | 29.3 | 16.5 | 158.4 |
| 193 | 61/WF | ** | 85 | 70 | 165 | 52 | 46.5 | 13.9 | 165.2 |
| 195 | 72/WF | MVD | 102 | 82 | 206 | 110 | 13.5 | 15.5 | 166.9 |

| Subject | | | | | | | |
|---|---|---|---|---|---|---|---|
| ID | A-II | C-III | E | B-100 | CETP | Statin RX | Risk Factors |
| 1 | | | | | | Y | HTN, LDL1+ |
| 3 | | | | | | N | FHx, LDL2+ |
| 7 | | | | | | N | FHx |
| 10 | 42.6 | 11.4 | 7.6 | 74 | 20 | Y (estrogen) | LDL(2+), FHX |
| 13 | | | | | | N | HTN, LDL1+ |
| 14 | | | | | | Y | FHx, tob+/−LDL3+ |
| 16 | | | | | | Y | none, LDL? |
| 24 | | | | | | N | FHx, tob+/−HTN, |
| 25 | | | | | | Y (Zetia) | tob+/−, HTN |
| 41 | 42.4 | 9.9 | 7.1 | 77.4 | 20.7 | Y | HTN, +/−tob, LDL3+ |
| 47 | | | 6.2 | | | N (estrogen) | HTN, LDL2+ |
| 49 | 46.7 | 11.6 | 6.2 | 94 | 31 | N (esterogen) | FHx, LDL1+ |
| 79 | 54.6 | 12 | 3.9 | 103.1 | 46.8 | N | FHx, +/−tob, LDL2+ |
| 84 | 32.6 | 12.2 | 2.6 | 67.7 | 13.7 | Y | HTN, DM, Tob, FHx, low HDL |
| 129 | 38.4 | 11.5 | 4.4 | 84.2 | 17.8 | N | LDL3+ |
| 141 | | | | | | Y, niacin | LDL1+, +tob |
| 143 | 37.8 | 9 | 1.7 | 63.4 | 28.3 | Y | HTN, tob+/−, LDL1+ |
| 146 | 42.9 | 12 | 4.3 | 82.6 | 39.9 | Y, estrogen | HTN, LDL3+ |
| 170 | 35.8 | 11.9 | 3.7 | 55.9 | 22.8 | Y, niacin | HTN, tob+, Fhx |
| 193 | 36.6 | 9 | 2.6 | 74.8 | 32.7 | Y | LDL2+ |
| 195 | 49.8 | 13.6 | 5.8 | 91.8 | 6.9 | Y | HTN, LDL2+ |

All lipid and apolipoprotein values specified as mg/dl. Lp(a) = lipoprotein-a, CETP is in pmol/ul/hour.
Abbreviations:
1) for race and gender: W = White, B = Black, H = Hispanic, F = Female, M = Male
2) for angiography: Nl = normal coronary angiogram, MVD = multivessel disease, CHD equiv. = CHD risk equivalent, ** = Normal stress test, *** = normal EKG and no symptomatic CHD or CHD risk equivalent.
3) for statin Rx: Y = yes, N = no, other notable lipid-altering medications are as specified .
4) Risk factors LDL 1+ = 100-129 mg/dl, 2+ = 130-160 mg/dl, 3+ = >160 mg/dl, ? = unknown, HTN = hypertension, Tob = tobacco; +/− = hx of; + = current, DM = diabetes mellitus, FHx = positive family history of CVD

### Apolipoprotein, Lipid and CETP

Apolipoprotein levels including A-I, A-II, B, C-I, C-III and E were measured in the Lipid and Lipoprotein Laboratory at the Oklahoma Medical Research Foundation (OMRF). ApoC-I was determined using the electro-immunoassay procedure of Curry et al., Clin. Chem., 27:543-548 (1981). All other apolipoprotein concentrations were determined by the immuno-turbidimetric procedure of Riepponen et al., Scand. J. Clin. Lab. Invest., 47:739-744 (1987), using corresponding monospecific polyclonal antisera. Lipid and lipoprotein-a levels were analyzed by standard methods in the Clinical Chemistry Laboratory at Scott & White. Cholesteryl ester transfer (CETP) activity was measured by Roar Biomedical Inc. (Calverton, N.Y.) utilizing commercial assays (RB-CETP) in a subset of subjects.

### Lipoprotein Subclass Characterization and Isolation

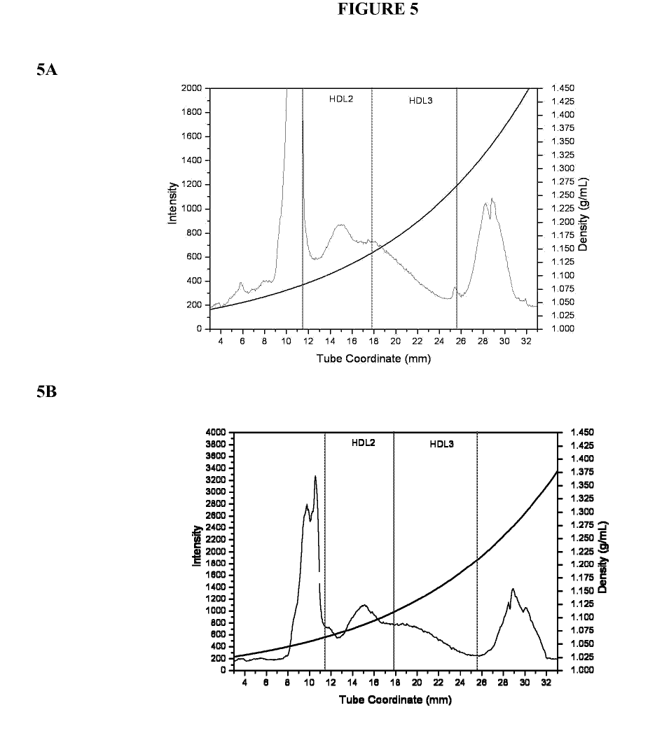

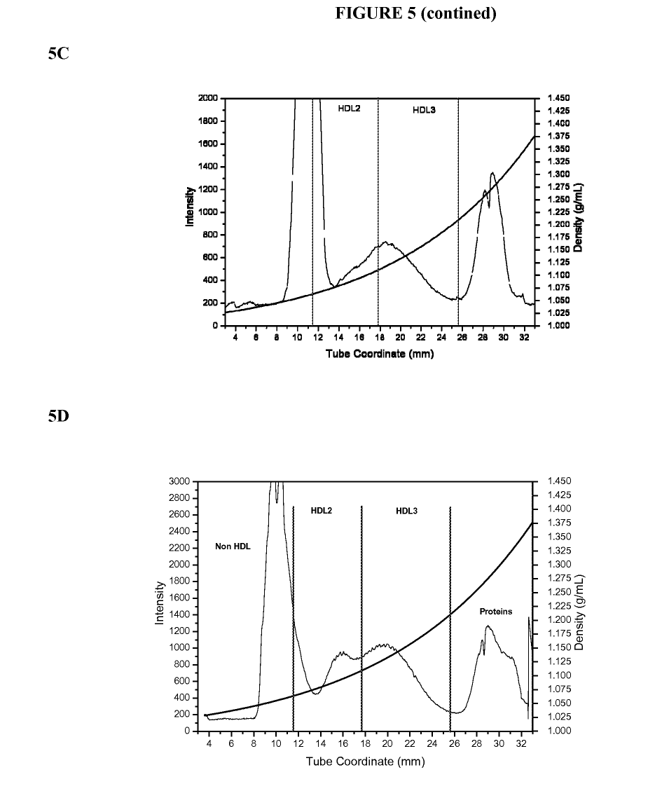

The lipoprotein subclasses were characterized and isolated by Density Gradient Ultracentrifugation (DGU) using a novel metal-ion ethylene diamine tetraacetic acid (EDTA) density-forming solute according to a procedure previously described (Johnson J. D. et al., Anal Chem., 77:7054-7061 (2005); Hosken B. D. et al., Anal Chem., 77:200-207 (2005); Espinosa I. L. et al., Anal Chem., 78:438-444 (2006)). Initially a self-forming sucrose gradient was used to characterize the lipoprotein particle density profile from infant cord blood containing apoC-I enriched HDL (Kwiterovich P. O. Jr. et al., JAMA, 293:1891-1899 (2005), but subsequently found that a variety of heavy-metal salts provided superior resolution of the various lipoprotein particles and the component subclasses. In addition, these salts were more easily removed than sucrose from the isolated lipoprotein particles. For the experiments described below, 0.18 M NaBiEDTA was used to screen samples for those with a prominent distribution of more buoyant HDL particles but 0.3 M Cs₂Cd(ethylenediaminetetraacetic acid (EDTA)) was used to isolate the HDL subclasses because this solute provided superior resolution of HDL₂ and HDL₃ subclasses and a better separation of HDL₂ from the lower density lipoprotein particles (LDL and lipoprotein-a), although it did not resolve LDL from very low density lipoprotein (VLDL). Serum (60-200 μL) was mixed with 1100 μL 0.3 M Cs₂Cd(EDTA), Sigma-Aldrich Corp. (St. Louis, Mo.) and 10 μL 6-((N-(7-nitrobenz-2-oxa-1,3-diazol4-yl)amino)-hexanoyl) sphingosine (NBD C6-ceramide), Molecular Probes (Eugene, Oreg.), in DMSO (1 mg/mL) EM Science (Gibstown, N.J.) in a 1.5 mL polycarbonate ultracentrifuge tube. Samples were spun for 6 h at 120,000 rpm at 5° C. in a Beckman Optima TLX-120 Ultracentrifuge equipped with a 30° fixed angle TLA 120.2 rotor, Beckman Instruments, (Fullerton, Calif.). The lipoprotein density distribution is imaged and converted into a spectra pattern using the fluorescence signal from the lipophilic NBD C6-ceramide (Henriquez R. et al., Atheroscler. Supplement, 7:587-588 (2006)). To isolate the lipoprotein fractions, the UC tube was immediately immersed in liquid nitrogen after ultracentrifugation; the lipoprotein fractions were excised from the frozen tube by slicing at predetermined cut points for the HDL₂ and HDL₃ subclasses (as noted by the vertical lines in FIG. 5). Each disc was thawed and filtered using a 30,000 molecular weight cutoff filter, Millipore (Billerica, Mass.) to remove low molecular weight components including the EDTA solute from the lipoproteins, and then passed through a 0.2 μm sterile filter (Millipore; Billerica, Calif.) to remove microbes and particulate matter.

### MALDI-TOF Mass Spectrometry

HDL fractions were desalted and delipidated as previously described (Watkins L. K. et al., J. Chromatogr. A., 840:183-193 (1999); Johnson J. D. et al., Int. J. Mass. Spectrom., 268:227-233 (2007); Moore D. et al., Biochem. Biophys. Res. Commun., 404:1034-1038 (2011) using a C18 cartridge, Phenomenex (Torrance, Calif.) and an equal volume of 0.1% trifluroacetic acid in water Sigma-Aldrich Corp. (St. Louis, Mo.). Mass spectra were obtained for 7 individuals with CHD, one individual without CHD but with a markedly apoptotic effect of HDL on ASMCs and all 8 subjects without CHD using an Applied Biosystems Voyager-DE STR (Foster City, Calif.).

### Fluorescence Microscopic-Quantitative Assay of Aortic Smooth Muscle Cell (ASMC) Apoptosis

The same procedure described in detail by Kolmakova et al., Arterioscler. Thromb. Vasc. Biol., 24:264-269 (2004) was used. In brief, 10³ human ASMC (Cambrex, Walkersville, Md.), were grown on sterilized glass cover slips in 6-well trays and treated with 2.5 μL of the various lipoprotein fractions per ml of medium. After 24 hour incubation, the medium was removed and cells were fixed and stained. The average ApoA-I concentration in the HDL₂ and HDL₃ fractions was ˜25 mg/dL and ˜40 mg/dL, respectively.

### Statistical Analyses

Correlations between apolipoprotein levels and HDL-C were carried out using the Pearson Correlation Coefficient. Apoptosis assays were performed in triplicate. Values were expressed as mean±SD. Student t test was used to evaluate the statistical significance of data. P<0.05 was considered as significant.

### Example 8

### Apolipoprotein, Lipid, and CETP Levels

Complete apolipoprotein levels and lipid levels were measured in a subset of 11 subjects (7 with CHD) (Table 2). The mean apolipoprotein values, standard deviations and normal values established for this subset of patients were:

ApoA-I, 156.5±11.4 mg/dL (normal 110-170 mg/dL),

ApoA-II, 41.8 mg/dL±6.6 mg/dL (normal 35-65 mg/dL),

ApoC-I, 12.8±2.7 mg/dL (normal<10 mg/dL),

ApoC-III, 11.3±1.4 mg/dL (normal 6.5-11.5 mg/dL),

ApoE, 4.5±1.9 mg/dL (normal 5.0-11.0 mg/dL) and

apoB, 80.0±14.0 mg/dL (normal 71-117 mg/dL).

The only apolipoprotein value outside the normal range was a higher apoC-I level. ApoA-I and apoC-I levels were measured for all samples except subject 141; there was a notably higher mean apoC-I level in the 8 subjects with CVD compared to subjects with angiographically normal coronary arteries (14.4±5.64 vs 7.9±1.0 mg/dL) while apoA-I levels were nearly the same (155.2±11.4 vs 156.8±12.8 mg/dL). The Pearson correlation coefficient of apoC-I with HDL-C was 0.72, P=0.002. Among the 8 subjects with CHD, the Pearson correlation coefficient improved to 0.96, P=0.0006 and was equal to the correlation with apoA-I. The correlation was not improved by using the ratio of apoC-I/apoA-I or the ratio of apoC-I/(apoA-I+apoA-II). No other correlations between apolipoprotein values and HDL-C were statistically significant with P<0.05. The mean CETP activity for 11 subjects was 25.4±11.4 pmol/μl/hour and 18.6±6.8 pmol/μl/hour for the subjects with CHD. These values are lower than most reported values; for example, in 855 adults participating in the Multi-Ethnic Study of Atherosclerosis, CETP activity ranged from 35.1-55.9 pmol/ul/hour (Tsai M. Y. et al., Atherosclerosis, 200:359-367 (2008)), and for 1,978 individuals in the Framingham Heart Study the mean CETP activity was 149±6.8 85 nmol/L/hour (Vasan R. S. et al., Circulation, 120:2414-2420 (2009)). CETP activity was best correlated with apoA-I (r=0.55, P=0.0619 for all subjects). There was no significant correlation between apoC-I and CETP activity—a finding also reported in the initial report by Kwiterovich et al., JAMA, 293:1891-1899 (2005).

### Example 9

### Lipoprotein Subclass Analyses

Using Cs₂Cd(EDTA) density gradient ultracentrifugation (DGU), the presence of a prominent distribution of HDL₂ particles was found. The HDL₂ particles were generally associated with an increased stimulation of ASMC apoptosis, especially if the HDL-C level was high (>60 mg/dL) as seen for subject 10 in FIG. 5A. Because the serum samples were drawn from a library of archived samples, it was investigated whether long-term sample storage (up to eight years at −80° C.) had any influence on the density profiles for 5 subjects with CAD and 3 subjects without CAD. Patients selected did not have any significant changes in their medications or medical history during this time. Although there were some minor changes in lipid levels with time, the lipoprotein density profiles were qualitatively independent of storage time. The comparison profiles for subject 10 are shown in FIG. 5A (archived serum) and 5B (fresh serum). A more Gaussian distribution of HDL particles is typical for most subjects as demonstrated by the DGU profile for subject 3 in FIG. 5C but in one case, the profile showed a more prominent HDL2 distribution as shown in FIG. 5D. The cut points for the HDL₂ fraction were defined by d=1.06-1.11 g/mL while HDL₃ fraction was defined as d=1.11-1.18 g/mL as represented by the vertical lines in the DGU spectra shown in FIG. 5. Although there are slight differences in the lipoprotein particle density distributions from different patients, these constant and predetermined cut points were used to excise the HDL₂ and HDL₃ fractions for the experiments rather than isolating fractions based on points of inflection in the DGU profile.

### Example 10

### Matrix Assisted Laser Desorption/Ionization Time-of-Flight (MALDI-TOF) Mass Spectrometry (MS) in Subject with and without CAD

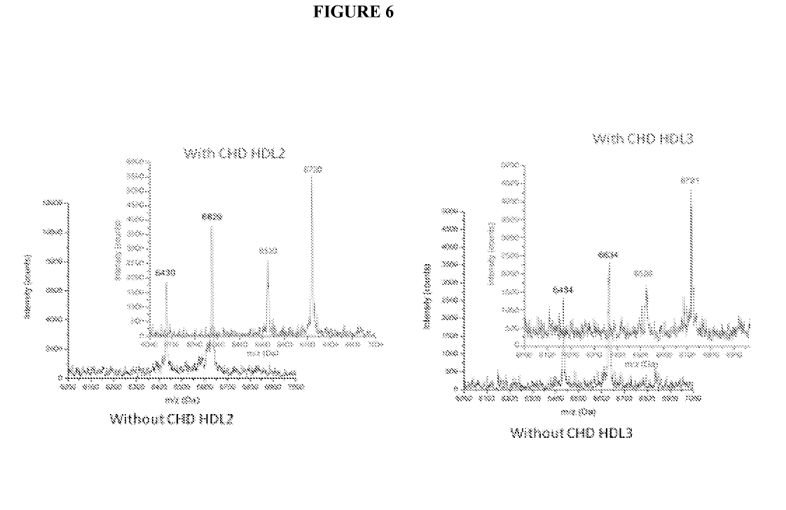

MALDI-TOF mass spectra were measured in a subset of samples. All subjects with a normal coronary angiogram exhibited a characteristic doublet pattern associated with apoC-I at m/z 6632.52±1.98 and a truncated form, apoC-I′ (ApoC-I minus N-terminus Thr-Pro) at m/z 6432.95±8.23 (Johnson J. D. et al., Int. J. Mass. Spectrom., 268:227-233 (2007); Moore D. et al., Biochem. Biophys. Res. Commun., 404:1034-1038 (2011); Bondarenko P. V. et al., Int. J. Mass. Spectrom., 219:671-680 (2002). These ions were also observed in the MALDI-TOF mass spectra of infants with apoC-I-enriched HDL (Kwiterovich P. O. Jr. et al., JAMA, 293:1891-1899 (2005)). An unexpected finding that was recently reported was the replacement of these characteristic ions in the mass spectra of 7 subjects with multivessel CAD (subjects 10, 41, 84, 143, 146, 170, 195) and 1 individual (subject 49) with apoptotic HDL by peaks shifted, on average+90 Da at m/z 6520±16 and 6721±8 that we believe represent a new mutation of apoC-I and its truncated form (Moore D. et al., Biochem. Biophys. Res. Commun., 404:1034-1038 (2011)). Comparative mass spectra for one subject with and one subject without CAD are shown in FIG. 6.

### Example 11

### HDL₂ Induces Apoptosis in Human Arterial Smooth Muscle Cells (ASMC)

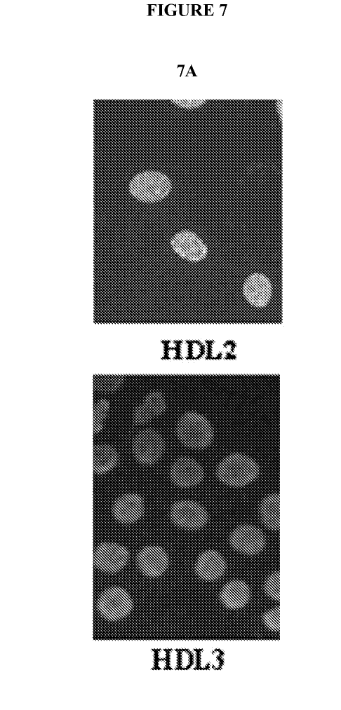

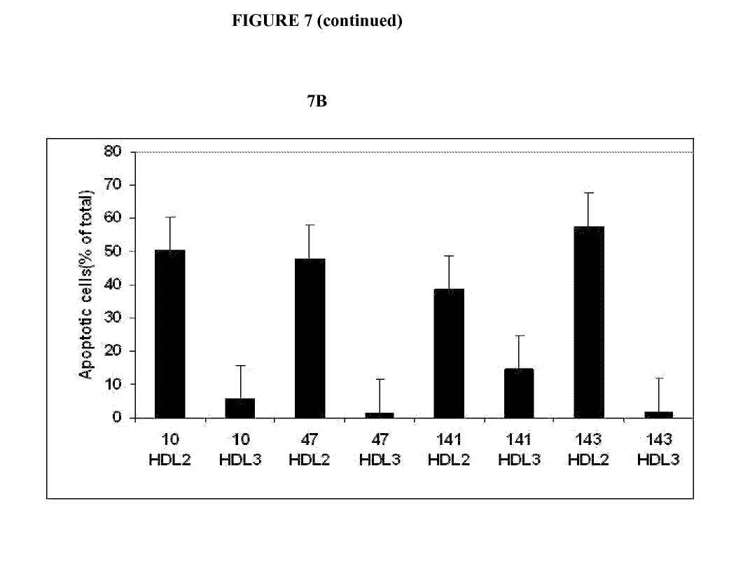

The effect of HDL₂ and HDL₃ lipoproteins from four representative subjects with CAD (subjects 10, 141 and 143) or CHD risk equivalent (subject 47) on human ASMCs was examined and is shown in FIG. 7. The DGU profile was characterized by a prominent HDL₂ distribution for all 4 subjects. The HDL-C was >60 mg/dl in 3 of the 4 subjects. The HDL₂ fraction stimulated apoptosis in up to 58% of ASMCs. The HDL₃ fraction from the same subjects had a markedly reduced effect ranging from 1.3% to 14.8% apoptosis. The magnitude of this effect is, on average, somewhat higher than that observed for apoC-I depleted HDL from cord blood but Kwiterovich et al. noted that apo-C-I is distributed throughout all HDL subclasses (JAMA, 293:1891-1899 (2005)). In many respects, the HDL₃ subclass serves as an internal control that suggests that the apoptotic affect is influenced primarily, if not exclusively, by the HDL particle rather than any other experimental variable. A concern existed that contamination of the HDL₂ fraction by high density apoB-containing lipoproteins, especially dense lipoprotein-a, may increase the extent of apoptosis, but no correlation was found with the lipoprotein-a level and the extent of apoptosis. Intentional mixing of the apoB-containing fraction with HDL₂ did not affect the extent of apoptosis.

### Example 12

### Correlation of ApoC-I Isoform with Apoptosis

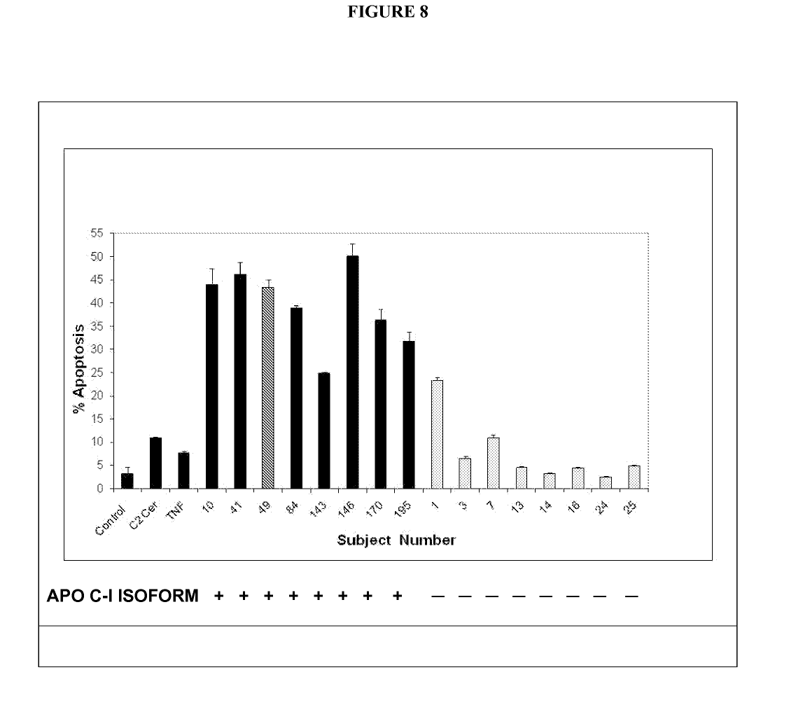

The effect of HDL₂ on ASMC apoptosis for 4 additional subjects with CAD, one without clinical symptoms of CAD and with a normal stress test prior to the blood draw, and 8 subjects with a normal angiogram was tested and is shown in FIG. 8. Fresh serum used for subjects 10 and 143 showed some decrement in apoptosis. However, HDL₂ isolated from a third serum sample for subject 10 induced apoptosis in 63±0.1% of cells (data not shown) indicating that there is some variability in the extent that the HDL fraction induces apoptosis. The extent of apoptosis induced by HDL₂ in subjects without CAD was markedly reduced compared to subjects with CAD; only subjects 1 and 7 had a higher extent of apoptosis than that induced by TNF-α. There were no characteristics (i.e., higher apoC-I or lower apoA-I/apoC-I levels, race, gender, risk factor burden or medications) that would distinguish these two subjects from the other controls, although as shown in FIG. 5, subject 1 had a more prominent HDL₂ distribution. This was not observed for subject 7. Overall, HDL₂ isolated from individuals with CAD or subjects without CAD but with an elevated apoC-I level induced a 14- to 25-fold enhanced apoptosis compared to the control. FIG. 8 also emphasizes the striking absence of the new apoC-I isoform in the mass spectra of the subjects without CAD. Although the association of apoptosis with the presence of the mass-shifted apoC-1 isoform is strong, the results obtained for subject 1, and to some extent subject 7 which demonstrated some degree of apoptosis but no mass-shifted apoC-1 isoform, suggest that there are components of HDL₂ particle contributing to apoptosis that do not involve this isoform. The results of exposing the HDL₂ and HDL₃ fractions from the remaining 3 subjects listed in Table 2 who did not have evidence of symptomatic CAD on ASMC apoptosis are as follows:

subject 79: HDL₂ 55±0.04%, HDL₃ 8±0.04%;

subject 129: HDL₂ 48±0.11%, HDL₃ 25±0.06%;

subject 193: HDL₂ 69±0.01% HDL₃ 36±0.09%.

Cells exposed to the HDL₃ fractions demonstrated significantly less apoptosis than the HDL₂ fraction but this effect was somewhat variable. We believe that slightly different excision points superimposed on variations in the overall HDL distribution may contribute to this effect. As can be seen in FIG. 5, the cut points that were selected to excise the HDL₂ and HDL₃ fractions resulted in some overlap of the various lipoprotein species resulting in contamination of the HDL₂ subclass with HDL₃ or vice versa depending on the actual DGU particle distribution. The cut points were chosen based on prior work and for consistency and did not vary from sample to sample although slight variations in the overall particle density profile were observed.

### Example 13

### Effect of Neutral Sphingomyelinase (N-SMase) on HDL₂-Induced Apoptosis

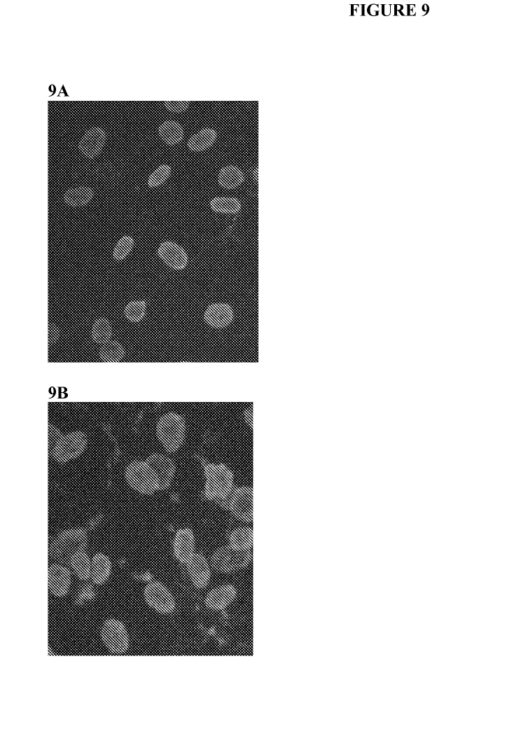

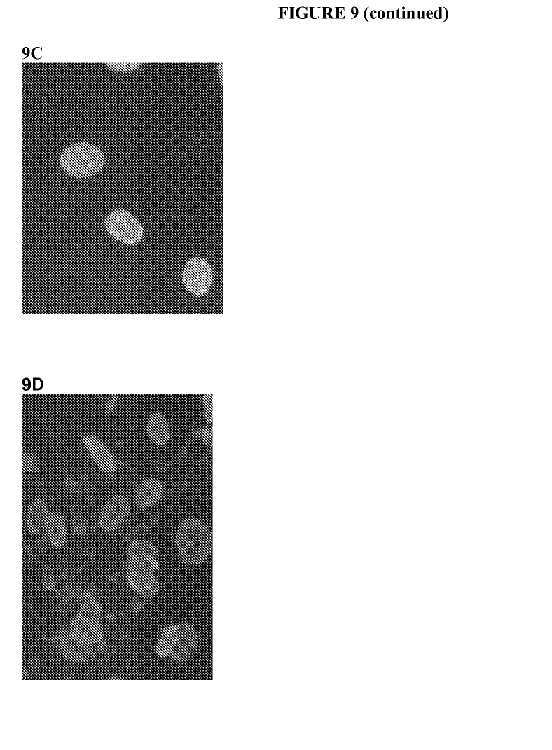

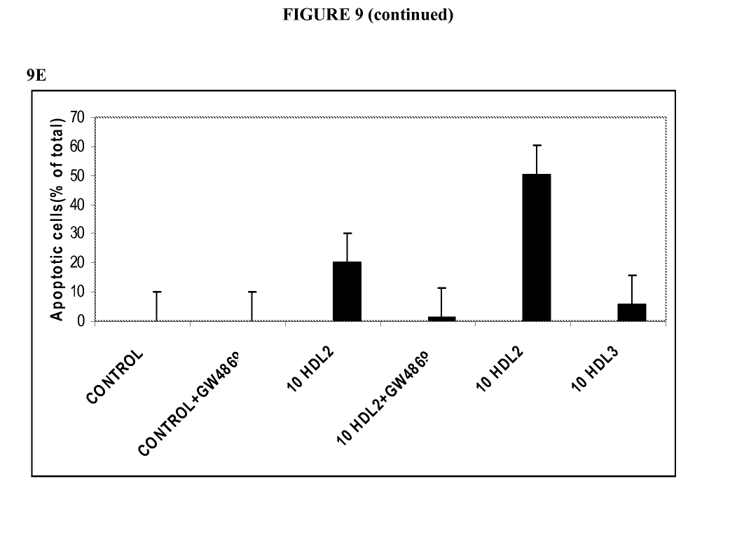

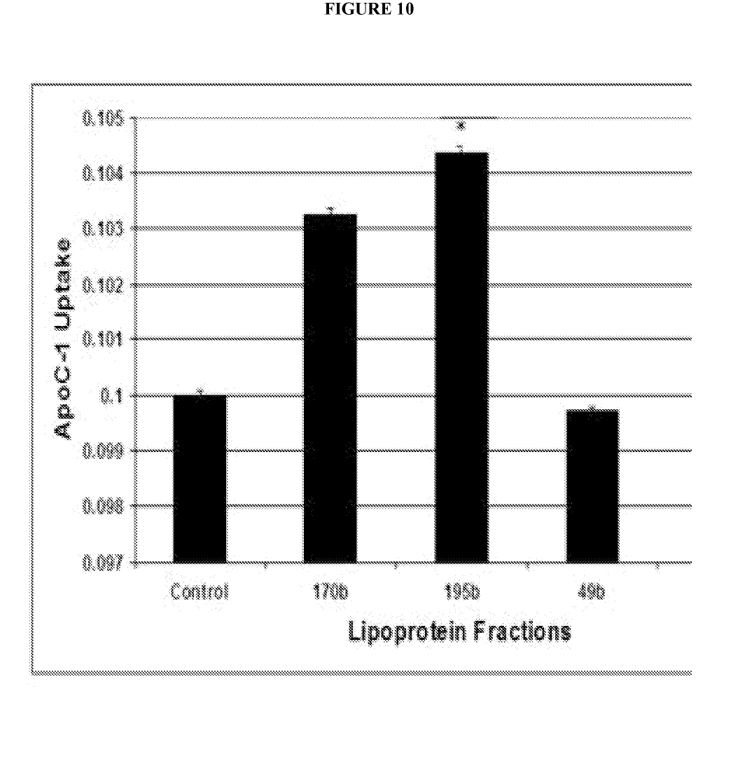

The effect of GW 4869 (20 umol/L), a noncompetitive inhibitor of N-SMase (Luberto C. et al., J. Biol Chem., 277(43):41128-41139 (2002)), on ASMC apoptosis mediated by HDL₂ from subject 10 was studied and the results are shown in FIG. 9. The addition of this inhibitor almost completely prevented HDL₂-induced apoptosis, reducing the number of apoptotic cells for HDL₂ from 20.1% to 1.19% in subject 10. The effect of HDL₂ on apoptosis was also dose-dependent: when only 1 μl of HDL₂ was used a 20.1% apoptosis of ASMC resulted as compared to ˜50% apoptosis when 2.5 μl HDL₂ from the same subject was used.

### Example 14

### Uptake of apoC-I and apoA-I in ASMCs

ApoC-I uptake in human ASMCs following incubation with HDL₂ fractions from subjects #170, 195 and 49 was examined To determine whether HDL containing apoC-I is taken up by ASMC, ASMCs were incubated with HDL fractions and pure apoC-I (the control), 2.5 μg/ml medium, for 24 hours followed by fixing. Next, the fixed cells were incubated with a primary antibody (1:250 dilution) against apoC-I (Academy Biomedical, Houston, Tex.) labeled with fluorescein isothiocyanate (FITC) (Sigma, St Louis Mo.). Fluorescence was measured using an ELISA plate reader/fluorimeter. All three HDL₂ fractions caused a high level of ASMC apoptosis as shown in FIG. 10.

### Example 15

### Effect of HDL2 on Nitric Oxide (NO) Release

One of the archetypal cardioprotective effects of HDL is eNOS activation resulting in NO formation (Li X. A. et al., J. Biol. Chem., 277:11058-63 (2002); Li X. A. et al., J. Biol. Chem., 280:19087-96 (2005)). This experiment was performed to explore the effects of apoC-I-enriched HDL on cultured HAECs to determine the extent to which NO and endothelial nitric-oxide synthase (eNOS) are affected by exposure to HDL₂ isolated from patients and shown to induce ASMC apoptosis. Although NO has many favorable effects on vascular cells, a cascade of unfavorable reactions can ensue when NO release is excessive, especially if other reactive species such as superoxide (O₂⁻) are present. This includes formation of peroxynitrite and further oxidative products such as nitrotyrosine (Xu et al., Am. J. Physiol. Heart Circ. Physiol., 290: H2220-H2227 (2006)).

Human aortic endothelial cells (HAECs) were incubated with HDL sub-fractions for 12 h from a subject with apoptotic HDL₂ (and CAD) and a subject with non-apoptotic HDL₂ (without CAD). The cells were then stained with 4′5′-diamino flourescein 2-diacetate (DAF) (Invitrogen/Molecular Probes, CA), a fluorescent probe that allows for real-time detection of NO. Following incubation cells were washed with phosphate buffered saline (PBS) to remove unbound dye and mounted on a drop of 10% glycerol in PBS and photographed by fluorescence microscopy.

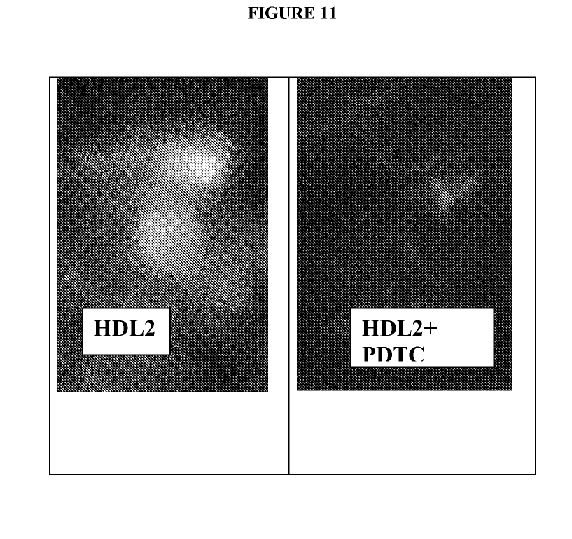

When HAECs were incubated with the same HDL₂ fractions that induced ASMC apoptosis (n=3 subjects) massive NO release was observed (FIG. 11) in contrast to the normal levels seen when cells were incubated with non-apoptotic HDL₂ (n=3 subjects). Pyrrolidine dithiocarbamate (PDTC) a membrane permeable anti-oxidant is widely used to study redox regulation of the intracellular signaling pathway has been shown to prevent IL-4-induced vascular cell adhesion molecule-1 expression (Lee Y. W. et al., Am. J. Physiol. Heart. Circ. Physiol., 284:H185-92 (2003)) and is also an inhibitor of NF-kB (Iseki A. et al., Biochem. Biophys. Res. Commun., 276:88-92 (2000)). Consistent with this, was that pretreatment of HAECs with PDTC markedly reduced NO release as shown in FIG. 11. This result was also confirmed by demonstration of markedly increased nitrite (the stable metabolite of NO) levels in the cell medium. Similar results were also obtained when HAECs were pretreated with NG monomethyl-L-arginine (NMLA); an inhibitor of NOS. Although increased levels of NO may induce apoptosis via reaction with superoxide to form peroxynitrite, such reactions were not observed with HDL₂ incubation. In contrast, the apo B100-containing lipoprotein fraction from the same subjects (n=3) did not induce NO release but did result in HAEC apoptosis.

### Example 16

### Effect of Different Lipoprotein Fractions on ICAM-1 Expression by HAECs

This experiment was performed to explore the effects of apoC-I-enriched HDL on cultured HAECs, specifically to determine the extent to which ICAM-1 and monocyte adhesion are differentially affected by exposure to HDL₂ and HDL₃ subclasses isolated from the same patients and shown to induce ASMC apoptosis.

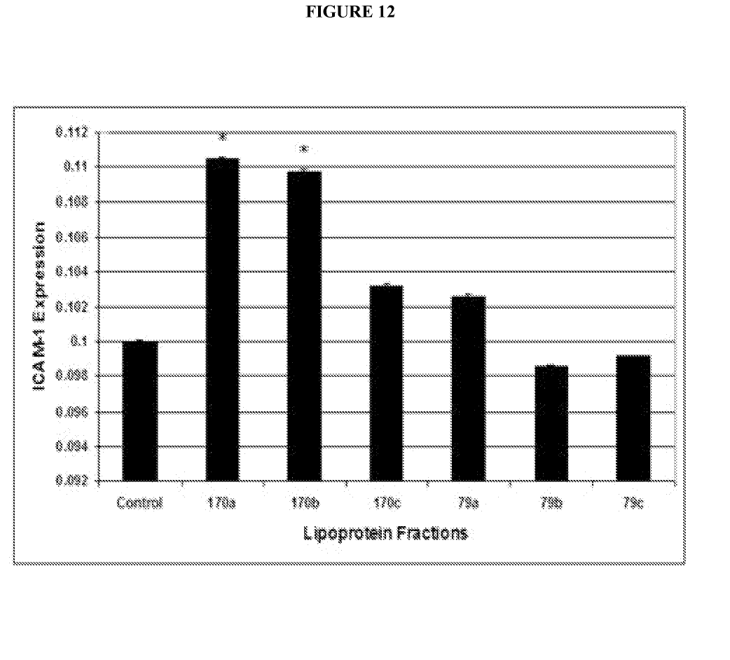

As shown in FIG. 12, the HDL₂ fraction but not the HDL₃ fraction from subject #170 induced as much ICAM-I expression as the apoB100-containing lipoproteins (LDL/VLDL). None of the fractions from subject #70 significantly affected ICAM-1 expression. The increased ICAM-1 expression was attenuated by pre-treating HAECs with NMLA and with PDTC. Since ICAM-1 expression was attenuated by an inhibitor of NOS (NMLA) and an inhibitor of NF-kB (PDTC) it suggests that both NO and NF-kB may be among several constituents in the signal transduction pathway leading to HDL₂-induced ICAM-1 expression.

Taken together, the results of Examples 15 and 16 support a model for the apoC-I-enriched HDL phenotype that is reminiscent of the proverbial “Trojan horse”: it is internalized and transcytosed by endothelial cells (Rohrer et al., Circ. Res., 104:1142-50 (2009)) where it exerts damage by increasing inflammation. Then, with access to the sub-endothelial space it can increase smooth muscle cell apoptosis resulting in destabilization of atherosclerotic plaque.

In sum, the data are supportive of a pro-inflammatory effect of apoC-I-enriched HDL on endothelial cells.

1.-95. (canceled)

96. A method of diagnosing or determining risk of developing coronary artery disease in a subject, comprising any one of:
(i) assessing a serum sample from the subject for presence of a first isoform of apolipoprotein C-I comprising 20 or more consecutive amino acids of SEQ ID NO:6, wherein the first isoform has a molecular weight of between 6.7 kD and 6.8 kD and presence of the first isoform in the sample indicates that the subject has coronary artery disease or is at increased risk of developing coronary artery disease compared to a subject without presence of the isoform;
(ii) assessing a serum sample from the subject for presence of a second isoform of apolipoprotein C-I comprising 20 or more consecutive amino acids of SEQ ID NO:7, wherein the second isoform has a molecular weight of between 6.5 kD and 6.55 kD and presence of the second isoform in the sample indicates that the subject has coronary artery disease or is at increased risk of developing coronary artery disease compared to a subject without presence of the isoform;
(iii) determining the molecular weight of apolipoprotein C-I isoforms of apolipoprotein C-I associated with high density lipoprotein2 (HDL₂) or high density lipoprotein3 (HDL₃) in a biological sample from the subject, and identifying the subject as being at risk for developing coronary artery disease if the apolipoprotein C-I isoforms associated with HDL₂ or HDL₃ in the sample have a molecular weight that is 50 daltons to 150 daltons greater than native apolipoprotein C-I (SEQ ID NO:6) or native apolipoprotein C-I′ (SEQ ID NO:7), respectively;
(iv) assessing whether a mass spectrum for HDL apolipoprotein C-I proteins recovered from the HDL₂ or HDL₃ fractions from a serum sample from the subject comprises a peak in the mass spectrum at between 6.7 kD and 6.8 kD, wherein presence of the peak indicates that the subject has coronary artery disease or is at increased risk of developing coronary artery disease compared to a subject without the peak in the mass spectrum at between 6.7 kD and 6.8 kD;
(v) assessing whether a mass spectrum for HDL apolipoprotein C-I or C-I′ proteins recovered from the HDL₂ or HDL₃ fractions from a biological sample obtained from the subject comprises a peak in the mass spectrum at between 6.5 kD and 6.55 kD, wherein presence of the peak indicates that the subject has coronary artery disease or is at increased risk of developing coronary artery disease compared to a subject without the peak in the mass spectrum at between 6.5 kD and 6.55 kD;
(vi) contacting HDL₂ or HDL₃ sub-fractions from a biological sample from the subject with an aortic smooth muscle cell, wherein the ability of the HDL₂ or HDL₃ sub-fractions to induce apoptosis of the aortic smooth muscle cell is indicative of the subject having or being at risk for developing coronary artery disease; or
(vii) contacting HDL₂ or HDL₃ sub-fractions from a biological sample from the subject with an endothelial cell, wherein the ability of the HDL₂ or HDL₃ sub-fractions to induce expression of nitric oxide and/or ICAM-1 at a greater level than HDL2 or HDL3 sub-fractions from a biological sample from a subject without coronary artery disease is indicative of the subject having or being at risk for developing coronary artery disease.

97. The method of claim 96, wherein the coronary artery disease is selected from the group consisting of atherosclerosis-associated plaque rupture, myocardial infarction, angina, and coronary ischemia.

98. The method of claim 96, wherein the subject is a mammal.

99. The method of claim 98, wherein the mammal is a human.

100. The method of claim 96, wherein in (i) or (ii), the first isoform of apolipoprotein C-I comprises 30 or more consecutive amino acids of SEQ ID NO:6 and the second isoform of apolipoprotein C-I comprises 30 or more consecutive amino acids of SEQ ID NO:7.

101. The method of claim 96, wherein in (i) or (ii), the first isoform of apolipoprotein C-I comprises 40 or more consecutive amino acids of SEQ ID NO:6 and the second isoform of apolipoprotein C-I comprises 40 or more consecutive amino acids of SEQ ID NO:7.

102. The method of claim 96, wherein in (i) or (ii), the first isoform has a molecular weight of between 6.7 kD and 6.75 kD and the second isoform has a molecular weight of between 6.51 kD and 6.53 kD.

103. The method of claim 96, wherein in (iii), (v), and (vii), the biological sample is a blood sample or a serum sample.

104. The method of claim 96, wherein in (iii), (v), and (vii), the biological sample was previously obtained from the subject.

105. The method of claim 96, wherein in (iii), the apolipoprotein C-I isoforms associated with HDL₂ or HDL₃ in the sample have a molecular weight that is 80 daltons to 100 daltons greater than native apolipoprotein C-I (SEQ ID NO:6) or apolipoprotein C-I′ (SEQ ID NO:7), respectively.

106. The method of claim 96, wherein in (iv), the mass spectrum comprises a peak between 6.7 kD and 6.75 kD or wherein in (v) the mass spectrum comprises a peak between 6.51 kD and 6.53 kD.

107. The method of claim 96, wherein in (iv) or (v), the apolipoprotein C-I protein measured by mass spectrometry is oxidized.

108. The method of claim 96, wherein in (vi), the aortic smooth muscle cell is a human aortic smooth muscle cell.

109. The method of claim 96, wherein in (vi) or (vii), the HDL₂ sub-fraction of the sample corresponds to a density range of 1.063 g/mL to 1.125 g/mL in a lipoprotein density profile and wherein the HDL₃ sub-fraction of the sample corresponds to a density range of 1.125 g/mL to 1.210 g/mL in a lipoprotein density profile.

110. The method of claim 96, wherein in (vii), the endothelial cell is a human aortic endothelial cell.

111. A method for identifying a compound that inhibits the development of coronary artery disease and/or is useful in the treatment of coronary artery disease, the method comprising:
(a) administering the compound to a subject exhibiting coronary artery disease;
(b) obtaining a high density lipoprotein (HDL₂) or high density lipoprotein₃ (HDL₃) sub-fraction from a biological sample from the subject; and
(c) determining the molecular weight of the apolipoprotein C-I isoforms in the HDL₂ or HDL₃ sub-fractions of the sample,
wherein the compound is determined to inhibit the development of coronary artery disease and/or be useful in the treatment of coronary artery disease if the molecular weight of the apolipoprotein C-I isoforms in the HDL₂ or HDL₃ sub-fractions of the sample is about the same molecular weight as apolipoprotein C-I isoforms in the HDL₂ or HDL₃ sub-fractions of an angiographically-normal subject.

112. The method of claim 111, wherein the coronary artery disease is selected from the group consisting of atherosclerosis-associated plaque rupture, myocardial infarction, angina, and coronary ischemia.

113. An isolated apolipoprotein C-I protein comprising 20 or more consecutive amino acids of SEQ ID NO:6 and having a molecular weight of 50 daltons to 100 daltons greater than the protein encoded by SEQ ID NO:6.

114. The protein of claim 113, wherein the protein has a molecular weight of (i) 80 daltons to 100 daltons greater than the protein encoded by SEQ ID NO:6.

115. The protein of claim 113, wherein the protein has a molecular weight between 6.7 kD and 6.8 kD.


SEQUENCE LISTING (7 sequences)

SEQ ID NO: 1 - DNA, 464 bp, Homo sapiens
ctccccagcctgataaaggtcctgcgggcaggacaggacctcccaaccaagccctccagcaaggattcagagtgcccctccggcctcgccatgaggctcttcctgtcgctcccggtcctggtggtggttctgtcgatcgtcttggaaggcccagccccagcccaggggaccccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatgaggacctgaagggtgacatcccaggaggggcctctgaaatttcccacaccccagcgcctgtgctgaggactccctccatgtggccccaggtgccaccaataaaaatcctacagaaaattcaaa

SEQ ID NO: 2 - DNA, 252 bp, Homo sapiens
atgaggctcttcctgtcgctcccggtcctggtggtggttctgtcgatcgtcttggaaggcccagccccagcccaggggaccccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatga

SEQ ID NO: 3 - DNA, 174 bp, Homo sapiens
accccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatga

SEQ ID NO: 4 - protein, 83 aa, Homo sapiens
MRLFLSLPVLVVVLSIVLEGPAPAQGTPDVSSALDKLKEFGNTLEDKARELISRIKQSELSAKMREWFSETFQKVKEKLKIDS

SEQ ID NO: 5 - protein, 26 aa, Homo sapiens
MRLFLSLPVLVVVLSIVLEGPAPAQG

SEQ ID NO: 6 - protein, 57 aa, Homo sapiens
TPDVSSALDKLKEFGNTLEDKARELIBRINKQSELSAKMREWFSETFQKVKELKIDS

SEQ ID NO: 7 - protein, 55 aa, Homo sapiens
DVSSALDKLKEFGNTLEDKARELIBRINKQSELSAKMREWFSETFQKVKELKIDS